(19) United States
(12) Patent Application Publication
(10) Pub. No.: US 2007/0067189 A1
(43) Pub. Date: Mar. 22, 2007

(54) Method and apparatus for screening, enrollment and management of patients in clinical trials

(75) Inventors: Ann S. Boris, Philadelphia, PA (US); John W. Houriet JR., Yardley, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Numoda Corporation

(21) Appl. No.: 11/522,803

(22) Filed: Sep. 18, 2006

Related U.S. Application Data
(60) Provisional application No. 60/718,221, filed on Sep. 16, 2005.

(51) Int. Cl. G06F 19/00; A61B 5/00

(52) U.S. Cl. 705/3; 600/300

(57) ABSTRACT

A computer-implemented method of tracking patient data in a clinical trial is provided. The clinical trial has one or more investigative sites which perform patient screening and enrollment for the clinical trial, one or more diagnostic sites which perform analysis on one or more patient diagnostic tests ordered by an investigative site and generate analysis results, and a centralized data center in electronic communication with the one or more investigative sites and the one or more diagnostic sites. Each investigative site is provided with a user interface display screen for allowing a user at the investigative site to enter data regarding patients who have been screened for the clinical trial and patients who have been enrolled in the clinical trial. The data from each of the investigative sites is electronically communicated to the centralized data center. Also, the analysis results from each of the diagnostic sites are electronically communicated to the centralized data center. The centralized data center consolidates the data and analysis results from each of the sites and provides one or more status reports regarding the patients for whom data and analysis results were received from the one or more investigative sites and the one or more diagnostic sites.

### CROSS REFERENCE TO RELATED APPLICATION

This application claims the benefit of U.S. Provisional Patent Application No. 60/718,221 filed Sep. 16, 2005 entitled “Method and Apparatus for Screening, Enrollment and Management of Patients in Clinical Trials.”

### COPYRIGHT NOTICE AND AUTHORIZATION

Portions of the documentation in this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND OF THE INVENTION

The number one challenge facing the clinical research industry is enrollment of the appropriate number of correct patients in a clinical study. This information is validated by the pharmaceuticals research and development industry. More than $2 Billion is spent on services to improve enrollment in clinical trials. From the largest pharmaceutical companies like Pfizer and Johnson & Johnson, to the smallest biotechs who are doing first-in-man studies, it is reported that enrollment is the number one challenge. Without a sufficient number of patients, the study to determine safety and efficacy, appropriate dosing, etc. for the new product or treatment will not be completed, and therefore cannot be submitted to the regulatory agency for approval to allow the product on the market. If the correct patients are not identified and screened speedily and the wrong patients are enrolled in the study (e.g. too sick for any new product to help them), then the study will not show any improvement in patient health as a result of the product. In this case, as well, the product will not be approved to market to the public. The pharmaceutical, biotech, medical device, and diagnostics companies that invent these new products need to conduct these studies correctly because their company's success depends on getting the product approved. Health care is waiting for new products to cure illness and/or improve the lives of patients with cancer, diabetes, Alzheimer's, etc. Patients are waiting for new cures and treatments and slow or incorrect enrollment causes problems for all these groups. McKinsey and Co., a global leader in consulting for the life sciences industry, reported from a survey of pharmaceutical company executives, that delays in clinical research are due primarily to the delays caused by poor patient enrollment. Therefore, it is important to the successful development of new drug research to be able to recruit, screen, and enroll the correct patients speedily.

Currently, there are several methods that attempt to solve this problem. Companies can advertise for patients. In fact, companies are spending increasing amounts of money and effort on advertising for patients to participate in clinical studies. This increases study and patient awareness, but does not guarantee that patients who contact the research centers are quickly and correctly screened for and enrolled into the appropriate study. The problem with this approach is that it is a great burden for the clinical investigative sites and staff to which these patients are referred to handle all the information and processes, screen interested participants, sort complex criteria for patient approvability, review a series of complex diagnostics, update their decisions based on changing criteria, keep adequate and accurate records, and track inconsistencies in informed and consent. These last two tasks are most often done incorrectly and are among the top five reasons the FDA reports that clinical trials fail (source Dr. Janet Woodcock, November 2002 interview with Drug Discovery and Development). All of these actions have to be performed within the window allowed for screening the patient into the study. With all this complexity and time involved, the site often cannot perform the tasks within the allotted timeline. The result is that the patients lose interest or their illness progresses, thereby precluding them from their approvability in the study, and the trial fails. In addition to delaying clinical development, this problem has increased clinical development costs.

Companies that sponsor clinical trials have attempted to alleviate this problem by conducting trials where there are larger populations of patients to evaluate. This includes conducting trials in Russia, Africa, Eastern Europe, and South America. There are many problems inherent in this plan to go to remote locations of the world and nations that are just developing their infrastructure. Clinical trials conducted in many diverse medical treatment cultures bring a serious increase in data discrepancies and differences among the studied population due to lack of standardization. One of the most serious concerns is that inappropriate patients are mistakenly enrolled into the study, causing the results of the study to be skewed, and therefore less reliable for the submission to the regulatory authorities. Costs can increase in these instances because although patients are more plentiful, reliable comparisons of diagnostic data, the compiling of the data into a single report and the analysis of the data, can be more costly.

Companies have also attempted to alleviate the problems of incorrect and untimely enrollment into the clinical studies by assigning specially trained medical staff to assist the sites in enrolling the correct patients into the trial. While this assists the investigator sites in identifying the correct patients, the problem is that it does not address the problem of the process not occurring speedily, and the process is even more costly. Often, the medical reviewer needs to spend time manually collecting all the information from various sources such as the investigative site, the patient, the diagnostic labs, etc. The medical reviewer does not have easy access (if any) to all the correct information to make a decision. Information about the patient is often changing; complex calculations of diagnostic tests are required, inclusion/exclusion criteria need to be reviewed and compared and contrasted, along with the version of consent signed by the patient. An additional problem is that the medical reviewer is unable to see trends with the information that if seen, would identify the need for the reviewer to request a change in criteria for inclusion/exclusion into the study. Moreover, the study team will not need to perform complicated projection calculations to identify if enrollment is on target. The present invention will compare the current numbers of patients being screened with that of the projections needed to meet the timeline and immediately message the team that more investigator sites need to be added to the study.

Another attempt at solving the patient recruitment problem has been to set up dial-up services, web sites and web pages for patients to search out clinical trials for which they might qualify. The web sites post new information on clinical trials, and allow a patient to enter some information about themselves, and the web site attempts to match the information entered by the patient with the appropriate criteria for the clinical trial. The Michelson et al. patent application is an example of this method. This method has had a similar result as advertising for patients. Many more patients are made aware, however the patient must still contact the site and the site must perform the complex process of screening diagnostic tests, review of test results and matching current inclusion/exclusion criteria with the correct patient for the trial. The problem is that once again, the investigator site or the clinical trial staff is responsible for getting the complicated information together, and processing this information in a short amount of time. Companies, who sponsor clinical trials do not have any assurance that the patients attracted by the above costly methods to the investigator site, actually enroll in their own trial. The investigator may enroll the patient in a competing trial (e.g. there are over 300 competing trials being run currently on competing HIV drug candidates, and several sites may be engaged in 10, 15 or more trials at the same time.)

Another recent method for trying to solve the recruitment and enrollment problem is the establishment of companies who purchase multiple healthcare data and databases of patient health information. This information is then queried for patients that match a particular search criterion that matches the criteria required for a particular clinical trial. Once again the problem with this method is that the costly, lengthy, and complex screening process at the sites is not eased. In addition, the data regarding a patient's health, their address, and their availability for a clinical trial are changing so rapidly, that the data are not as broadly useful as hoped. There is not tracking and review available from easily accessed reports generated real-time to assess what is actually transpiring at those sites in the screening and enrollment process. The patent application for data mining from Siemens is an example of this approach. Other examples of this type of thinking and this approach are described in U.S. Patent Application Publication No. 2002/0002474 (Michelson et al.); U.S. Patent Application Publication No. 2002/0099570 (Knight); U.S. Pat. No. 6,839,678 (Schmidt et al.); and U.S. Patent Application Publication No. 2003/0130871 (Rao et al.). Numoda has identified that enrollment in clinical trials has more difficult and complicated problems and logistical challenges than better recruitment will solve. Better recruitment only means that more patients in the world will hear about clinical trials for their illnesses. This does not ensure that the correct patient will be enrolled in the trial. There is ample data verifying that decisions to enter a trial are made at the point of contact with a physician at the site. Better recruitment means patients can be tentatively matched with trials that are appropriate to their disease. However, during the complex screening process it may still be found that the patient does not meet all the characteristics for the trial.

The problem facing the industry is that trials are becoming more and more complex. Sites that will potentially enroll patients into the study are scattered all over the world and are subject to additional regulations, apart from the regulatory bodies that approve new drugs. There are many suppliers, scattered all across the globe, that play a very important part in screening patients for enrollment in a trial. The members of the study team that will manage and monitor the enrollment processes for a trial are also scattered around the globe, in places apart from the suppliers and investigator sites. Another problem is that the study team members, as well as all other groups, are ‘siloed’—separated into independent groups that have difficulty communicating and exchanging information. The logistical challenges of information exchange, keeping track of the changing requirements for enrollment, and the logistical problems of multiple groups in different time zones are not solved by the prior art.

The following actual example has been de-identified for confidentiality purposes. The use of the present invention in this clinical trial reduced the screening to enrollment ratio from 5 to 1, down to 1.4 to one. Before use of the present invention, these specific trials required the screening of 5 possible patients, to obtain one enrolled patient. This improved accuracy in screening has reduced the cost of screening patients and the time it takes to enroll the appropriate number of patients in a clinical trial. The regulatory agencies will now more quickly receive a report on the trial for analysis of the new treatment. Improvement in the accuracy of patients enrolled will more likely result in receiving the correct patient examples. Life-saving treatments are more likely to be approved in a timely manner.

FIG. 1 is a diagram that depicts the current, complex and time consuming process from the recruitment and prescreening process seen in ‘A’, to the informed consent process seen in ‘B’, to the Screening and Enrollment Process in ‘C’. The process of finding patients for the study is not shown on this diagram but can be seen in the patents and patent applications that are referenced as prior art. FIG. 1 shows how the process for 200 patients that have been recruited ends in only 12 patients enrolled. The process is very linear, with multiple points of decision, multiple different staff involved, and very little information including integrated information available, if any, about why the patients are not given the informed consent for the trial, why patients did not sign the informed consent, and why patients failed screening for the trial. In ‘B’, the receptionist does not record that the patient will not consider signing the informed consent because bus fare is not supplied for him. Of the patients that were given the informed consent, examiner1 records the reason that a patient can't afford bus fare. Examiner #2 does not record her patient's reason for not signing the informed consent, which is the same reason as the patient seen by examiner #1. Still further in the process, although examiner #1 records the need for bus fare, this information is never reported to the monitor for the study. There is not much information available on why only 185 of the 200 patients that were recruited in ‘A’ (or who have been identified by their doctor) as a potential patient for a clinical trial are asked to come in to the investigator site. This trend continues in ‘B’, where busy sites must manage one hundred patients and then collect the consent of the 60 patients that agree to be considered for the trial. The sites focus more time and effort in collecting the informed consent. Sites do not always take the time to collect, record and report the reasons that the patients do not consent, and this information is not available to the people who are managing the trial. Often, reasons are simple and support adjustments such as paying for bus fare for the patient, may enable the patient to partake in the study. In ‘C’, more detailed health information is collected on the patient. Prior to signing the informed consent, it is not allowable to get these details that involve the collection of diagnostic specimens or performing a physical exam. In ‘C’, depicting the process for more in-depth examination to establish the patients' eligibility for the trial. However, in early screening, the examiner may find that the patient is excluded from the trial because the patient does not use birth control. The examiner might not record this information, however it is usually recorded at the site. Yet, if the examiner does not report this information, it is not available to the entire study team. It would be valuable to the study team to know all information about why patients are not enrolled in the study. Analysis and trending of this information allows the study team to take a proactive role to increase the number of patients enrolled into the study. Even when the site records the information, this information is still not available to the entire study team or other investigator sites unless a visiting clinical monitor(s) reports this information during a routine visit to perform other tasks at the site. The site must actively record the information, and then remember to supply that information to the monitor during a visit. The study team must then be proactive in reviewing and analyzing the information to identify problems that adversely affect the screening and enrollment process at this single site and at multiple sites. The informed consent process and the screening and enrollment process are more complicated than depicted in FIG. 1.

FIG. 2 depicts the process that will occur for a patient that is willing to sign the informed consent that will then be co-signed by an investigator that is an approved doctor in the study. Even when a patient is willing to sign informed consent, the process of getting a consent signed is logistically challenging to the site. Sites must remember all the information available that pertains to screening the patient, for any of the studies being conducted at that site. Sites often partake in many different studies and companies who sponsor the studies are ‘fighting’ for the patient to be ‘consented’ into their study. If the study is very complex and the screening criteria are difficult to remember and understand, the site may simply screen the patient into a less complicated study. FIG. 2 shows that the site must remember that version 2 of the informed consent is the most current version and that the patient must sign this version. It further depicts that the site must then decide which of the protocol versions for that study are to be followed for this particular patient. When the site decides to screen the patient into a particular study, the site must remember/review all of the information pertaining to the requirements for the particular study. There are many procedures that are followed as part of a particular protocol:

Inclusion/Exclusion criteria, ECGs, Labs, CRFs, for the site to perform, such as diagnostic tests on the patient. The site must use the most current version of the protocol and all the required follow on procedures. The most current protocol inclusion/exclusion criteria are used to verify that the patient who signed informed consent meets the currently deployed criteria for the study. If the proper version of the protocol and procedures are not followed, then the patient will be considered in violation of the protocol. Often information about a protocol violation is not discovered until far into the trial. This patient's data, despite all the efforts made to enroll and treat them in the study, will not be part of the final study report. As seen in FIG. 2, studies often have several versions of the protocol. Only the most recent protocol that has been approved by an independent review board will be used, at that point in time, for that site. Other versions of the protocol may be available but not approved for each site. This is a common occurrence where global sites require additional translations and paperwork that are not required in the United States. The site must remember all this information in order to choose the correct protocol to follow.

In addition, there may be allowable exceptions to these rules, and the exceptions must be carefully recorded, tracked and managed. If too many exceptions occur, this signals the regulatory bodies that another protocol amendment is required. If the sponsoring company for the trial allows exceptions, they need to have a policy in place for granting such waivers. Consider the example, where age range criteria for the study is 18-65 years of age and the policy for age is to calculate from the date that the informed consent is signed. A waiver might be given for a patient who will reach his 66^{th} birthday during the trial. The parameter for age is calculated from the date of randomization, rather than from the date informed consent is signed. The site may forget to ask for a waiver or miss the fact that the patient will need one, if the site does not remember the new criterion or if they do not calculate the date of birth against the correct date. They will have performed this process incorrectly. The site may also not remember that there is a policy for attaining a waiver (allowance) for this age range, because the new version of the criteria is in the process of being accepted by the sites' governing review board. The site must remember and implement all these steps in order to correctly enroll this patient. The site must also keep track that although a younger patient cannot enroll at this time, the site can call the patient when they reach the correct age or can get a waiver for approval in the study. Sites do not have the time or the staff to perform these functions with 100% accuracy, nor the ability to track the patient at a later time. This results in a screen failure for the study.

FIGS. 3 and 4 show the required schedule of procedures for two different protocol versions. It is easy to note that the changes are barely perceptible in a complex grid such as this. Row A of FIG. 3 has eight days marked with an ECG. FIG. 4 row A only has 6 days marked with an ECG. FIG. 4, row B and C were added to the protocol and it is important to note that the protocol does not point out that there are additions or changes to the protocol that the sites must consider when screening and enrolling patients. It is not hard to imagine that as studies get more complex, and changes are made to the protocol, that sites do not, or cannot, enroll patients into these studies.

### BRIEF SUMMARY OF THE INVENTION

An integrated, interactive and dynamic, screening and enrollment management system is provided that handles the logistics of information management for patients that have been recruited for a clinical trial. The present invention is an information management system and method for pharmaceutical, biotech, medical device diagnostics companies, clinical research organizations, recruitment companies, clinical investigation centers, registries, and marketing companies that simplifies, speeds, and improves the accuracy of the screening and enrollment process for clinical trial patients. The present invention performs real-time harnessing and consolidation of information and data from any and all systems, participants and groups that are part of the process (e.g. ECG, labs, specialty labs, IVRS, other diagnostic testing, clinical monitors, investigator sites, etc.). The present invention standardizes all the data from any system, and then reconciles the data against all other data within the system. All data are processed with algorithms that are specific to a trial protocol and accepted values, and with estimated metrics for the trial. The present invention sends the data that does not reconcile, into reports and then messages the appropriate party that an inconsistency exists. These messages and reports provide that party with a proposed solution to solve the discrepancy. The present invention also displays trends in the screening and enrollment and provides possible solutions. The present invention tracks patients that have not qualified based on a set of criteria, and display the criteria under which the patient will qualify.

The present invention is an integrated, interactive, screening and enrollment management system that solves the problems of screening and enrollment of patients that have already been recruited for specific trials that are being conducted at investigative sites. This is an advancement of the current methods. It takes the very complicated patient, site and external supplier processes that occur beyond the recruitment phase, and applies logistics management, processes study specific algorithms, collects of information from multiple systems, processes the information, and selectively pushes the appropriate information to investigators and study team. The present invention addresses the very complex, multidimensional, and logistical problems that have caused delays and failure of patient screening and enrollment in a clinical trial at the investigator site, after the process of recruitment. The present invention solves the complex logistics for handling recruited patients and then screening and enrolling patients correctly, and in a timely fashion, into the trial. It costs the sponsor of the trial a lot of time and money to recruit a patient, and this money is lost when recruited patients are enrolled in a competing trial for a different sponsor. The present invention solves that problem. While screening failure rates are often very high, and hundreds of patients, even thousands need to be recruited to get a few patients that are appropriate for screening and then five patients need to be screened to enroll just one correct patient, the present invention reduces the rates so that of every 1.4 patients screened, one patient is appropriate for the study. The present invention accomplishes this because it assists the clinical study site in more quickly and accurately identifying a recruited patient for screening and enrollment into a specific clinical trial. The present invention identifies, simplifies, streamlines, and immediately reports that a patient can be enrolled in the study. By reducing a complex and multi-step process that takes place at a busy doctors office or research center, where multiple trials and patient treatment are taking place, the sponsor can be assured that appropriate patients are enrolled in the study.

Where the prior art only improves the recruitment for a study by matching any patient's basic medical characteristics or historic characteristics with the several potential trials they may wish to join, the present invention easily and quickly provides the customized interfaces that are needed for an integrated, interactive approach to the problem. The present invention reduces the level of complexity of the logistics required after the recruitment process.

The present invention also reduces the complex processes that require the timely management of a number of logistical procedures such as lab tests, medical exams, information collection, signatures on documents, etc. Because of the present invention, the study staff need not be as familiar with the multiple tasks that need to be performed to enroll a patient and the multiple criteria for patient inclusion and exclusion in the trial. The present invention reduces the confusion of sorting between the multiple versions of criteria, multiple versions of tasks, all within specific, accepted dates for implementation of the criteria. The present invention makes it easier for the staff to collect and transport the correct diagnostic tests that must be processed (may be done by a separate department). After processing, the present invention makes test results immediately available, performs a review, and may interpret the tests to determine that the patient meets the appropriate version of criteria. The present invention automatically notifies the right person regarding this information.

The study staff also needs to interpret clinical criteria that are ambiguous. FDA reports that failure to follow the protocol as another of the top five reasons clinical trials fail. Staff must perform a different set of review for female vs. male patients, older vs. younger patients, etc. Study staff must also be knowledgeable of normal ranges, and the generic, trade, and class names for medications that would exclude a patient from the trial. All of these procedures and interpretations must be done within a window of time that is set by the criteria, adding an additional layer of challenge. Patients who do not meet the criteria are often not tracked, to establish if these patients might need to be reevaluated for the criteria, for example, at a later time. The present invention of an integrated, interactive, screening and enrollment management system solves the above problems and provides investigator sites with a suite of integrated electronic applications. This system includes, many functions and features that perform calculations, eliminating human error and speeding decision-making. Other features include the identification of missing tasks, and reporting these missed tasks. Patients that have been recruited for a trial are easily and quickly identified at the investigator site by a set of screening and enrollment tools at the clinical trial site. The patient clinical, diagnostic, demographic, and all other data needed for enrollment are managed, processed and tracked throughout the complex screening process, by means of these tools. The system reports on what change in the patient's health could allow them to be enrolled at a later time. The present invention provides an integrated view of the clinical data, the diagnostic data and the inclusion/exclusion criteria. And elicits and tracks the necessary consent forms for enrollment.

The present invention accomplishes another important step as well. The integrated data are now analyzed for all the investigator sites and tracked and trended for information on how enrollment rates will be affected based on projected changes in the design of the study, inclusion/exclusion criteria, and other parameters. All this information is reported to the wider project team and the sponsors for the study, and identifies the changes that can be made in these factors to improve the rates of patient enrollment for the study overall.

This system provides the clinical study team that manages all the investigator sites, with access to the patient clinical information that has been collected by all the investigator sites. This includes all the diagnostic results, and any version of the clinical trial protocol's inclusion/exclusion criteria. This integrated system performs complex processing for the total of screened patients to identify trends in enrollment. It also tracks changes in the patient diagnostic data or changes in the trial's inclusion exclusion criteria to identify patients who can be re-screened for enrollment in the trial. The present invention has eliminated errors made at the clinical investigator site, assuring that the appropriate patients are quickly enrolled. The present invention has also made an integrated view of the data available to the medical reviewer, to help perform the complex process of identifying the correct patient without the input of the medical reviewer. The study teams get trending information that assists them in more quickly and accurately estimating, in advance, that the current rate of enrollment at the clinical sites will not be sufficient for enrollment, and estimates how many additional sites are needed to reach the correct enrollment numbers.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

FIGS. 1-4 show prior art procedures for screening and enrolling patients in clinical trials.

FIG. 5 shows procedures for screening and enrolling patients in clinical trials in accordance with one preferred embodiment of the present invention.

FIGS. 6-18 show user interface display screens for implementing the procedures of FIG. 5 in accordance with one preferred embodiment of the present invention.

FIG. 19 shows a gateway integration process in accordance with one preferred embodiment of the present invention.

FIGS. 20-47 show additional show user interface display screens for implementing the procedures of FIG. 5 in accordance with one preferred embodiment of the present invention.

FIGS. 48-50 are data table structure diagrams for implementing the procedures of FIG. 5 in accordance with one preferred embodiment of the present invention.

### DETAILED DESCRIPTION OF THE INVENTION

In describing embodiments of the invention illustrated in the drawings, specific terminology will be used for the sake of clarity. However, the invention is not intended to be limited to the specific terms so selected, and it is to be understood that each specific term includes all technical equivalents which operate in a similar manner to accomplish a similar purpose.

The present invention is described in the context of a commercial embodiment implemented by Numoda Corporation, Philadelphia, Pa.

The present invention is an information management system and method for real-time, integrated, interactive, screening and enrollment management and reporting. There are several novel aspects to the present invention. It is available as a completely integrated and interoperable system, on a trial-by-trial basis. Unlike other systems that take many years to design, specify and build, the present invention works on a platform of configurable data capture objects, reporting objects, and messaging objects, making it easily configurable. An entire trial screening and enrollment system, with its integration and interoperability, can be set up in just a few weeks. It is also very flexible to changes, and it can be configured to import and export data via a gateway, to and from any supplier's database. The gateway can accept data in any format, from any supplier and the integrations can be set up within days. The quick set up and flexibility has only been achieved with the present invention, and is the key to the success of the present invention in the pharmaceutical, biotech, medical device, and diagnostics industries. The present invention can accommodate the different configuration and set up for any trial, in a therapeutic area, working with any vendor, and any communication medium. The present invention is also an advancement in ongoing consolidation and reconciliation of all the data from any integrated data source. Reporting is done after data has been processed for mismatches between data sources. This advantage allows any authorized person to access from a central database, in real time, the information from multiple vendor databases. Another important advancement is the immediate visibility provided by the present invention. Immediate, constantly current reporting of all the information is available; all in one place, and these reports can be accessed from any computer. This type of interoperability had not been considered possible in the life sciences industry, particularly because of the many multiple suppliers and systems and databases that take part in the enrollment process for a clinical trial. There is a movement in the industry to require that all involved parties work in a common format. This work to standardize across a single format has been going on for over 10 years, with no clear standard decided, and no agreement from suppliers to accept this standard. The present invention does not require a standard format, but accepts data in any format, maps the data correctly, processes discrepancies and provides information on where discrepancies exist.

Complexity and errors are reduced because the present invention imports, then matches information from external diagnostics suppliers such as labs, ECG, etc. Information from these suppliers is consolidated, and compared against the approved criteria for a specific investigator site. The data may also be consolidated with an algorithm that provides correct enrollment criteria information if the criteria is not specifically named but references for example, “normal”. Sites need to perform the correct, accepted calculations for age, and gender to arrive at a “normal” value. Reducing these inconsistencies across sites by providing the “normal” values reduces the approval of inappropriate patients into the study. Without the present invention, clinical and medical monitors must review all the incoming materials, perform calculations for appropriateness and be faced with making errors in their calculations. The present invention prevents those errors and logistically manages the calculation of normal ranges for age range, gender, race, etc. In addition, the entire study team receives trend analysis reports of diagnostic failures for the study and is presented with a percentage of improvement in enrollment, should the ranges be adjusted with a protocol amendment.

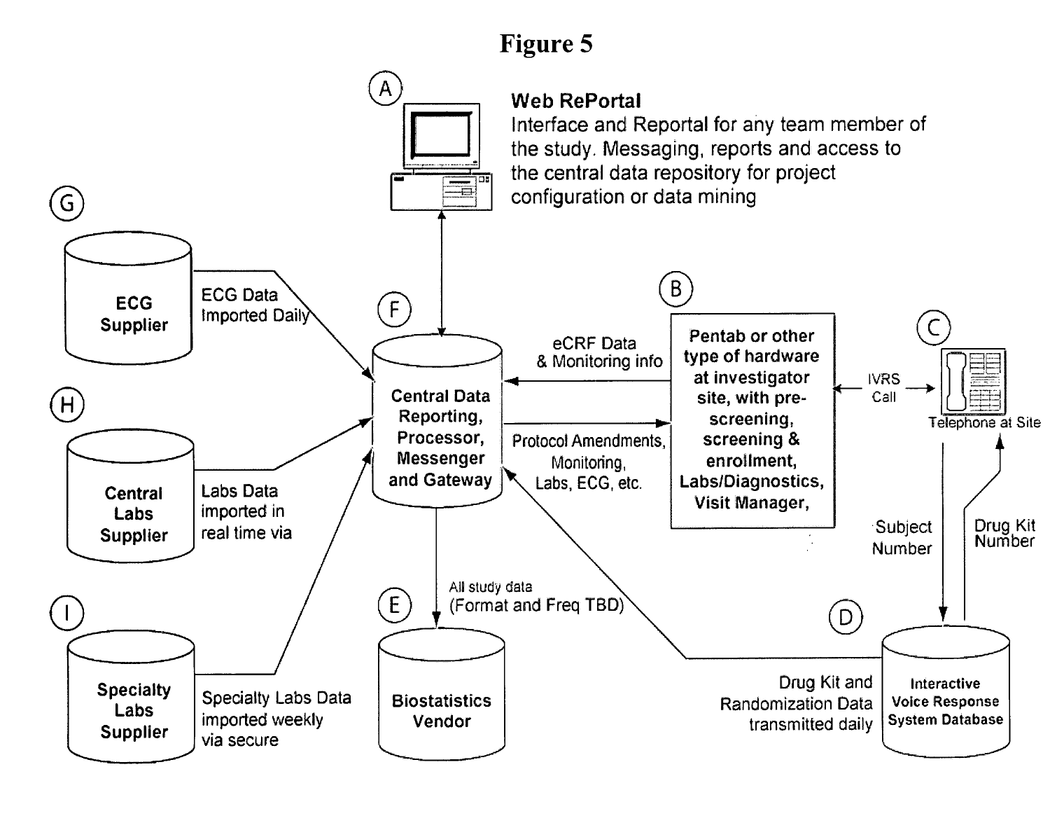

FIG. 5 shows the configuration of the system with a central data repository ‘F’ (“centralized data center”), connected in a spoke-like network. It is important to note that the network of connections to the lab ‘H’, ECG ‘G’, specialty labs ‘I’, and interactive voice response system ‘C’, are all systems or databases that reside outside of the corporate network in which the present invention resides. The present invention connects with and imports the data that is necessary to screen and enroll a patient successfully for a clinical trial. FIG. 5 ‘A’ shows a computer that is one of any number of computers that will connect to ‘F’, in order to set up the system for the trial, receive reports and messages, and access the data directly. The present invention will help the widely dispersed study teams obtain immediate information, from the widely dispersed suppliers, on the status of screening and enrollment. FIG. 5 ‘B’ shows how investigator sites are made part of the network and integrated into the process to both record and report information, and to receive information as well. A set of site tools, that are part of the present invention, is made available on the hardware at the site. These tools will record and report on enrollment for the site. These tools track and manage recruitment, prescreening, informed consenting, screening, and enrollment at the sites. The tool manages versions of the protocol and all required CRFs, labs, ECGs, specialty labs, inclusion/exclusion criteria, and any other information. The site would simply select a patient, and the present invention would display and prompt for recording only the information that is required at this particular time point, for this particular trial, for this particular patient. The present invention makes more information, more accessible, by more people, in order to help them with enrollment. The present invention not only provides information, it reports the information with suggestions to improve the enrollment for the trial. The present invention has caused an increase in the number of correct patients that are enrolled in the study, by having computers manage all the complex logistics of the enrollment process. The present invention has removed the time consuming, frustrating work of getting to the information that is needed to positively impact the enrollment of patients in a study. The present invention has also made it easier for the sites, whose job it is to perform all the complicated tasks to enroll the correct patient speedily.

The following example shows how the present invention is used in a clinical trial. A clinical trial protocol is written by a group of clinical and scientific experts, who define the desired endpoints for the trial, and the type of patients to be included or excluded in the enrollment of the trial. The experts decide the diagnostic tests and other procedures needed and the acceptable results for those tests or procedures, in order for a patient to be enrolled in a trial. This same group also changes the protocol during the course of the trial. They do this for many reasons; one important reason is to get the right number of correct patients enrolled in the trial. It will be a problem for the study if the criteria are so rigid that they exclude appropriate patients for the trial. When the protocol specifies the kind of patients and the number of patients that are to be included in the trial, a clinical study team will begin to set up the investigator sites, which estimate how many patients they can recruit and enroll in the study. The present invention provides an interface through a web reportal seen in FIG. 5 ‘A’. This interface includes a set of tools that allows a person to take the information that is available in the protocol, together with estimates from the clinical study team, and then set up the parameters and establish the appropriate algorithms that will be used to process data, to manage the logistics of screening requirements for the patients in the trial, and finally to establish the reports for the trial. Enrollment projections are usually established before a trial starts, and are based on the estimates that sites provide during their contracting process for the trial. It is important to enroll the appropriate amount of patients—not too many or too few. It is important to track what patients that sites are enrolling and at what rate, and identify sites that may not be able to enroll any, or just one—that too complicates site management. For example, a site may project that they need to screen 3 patients to enroll one patient into the study. They may also project that they can enroll one patient per month. With this information, the study team can project that 20 sites will enroll 180 patients in six months. This type of planning occurs in advance of the study start, and is established based on historical trends for particular sites, for similar studies. Some companies estimate that on average there is a 90% error rate in this process, as many factors are unknown before the study starts. During the study, new information on factors affecting the study can only be collected and analyzed if and when sites provide information. Monitors visit the site every 6-8 weeks and collect information, and/or IVR systems import reports, and/or diagnostic lab providers make available information on lab results for screened patients. When all this information is available, a team member is required to cross-reference, compare and provide an analysis of the differences between projected and actual enrollment. The present invention pulls together all the information, without collection and data entry and without human intervention to analyze the numbers.

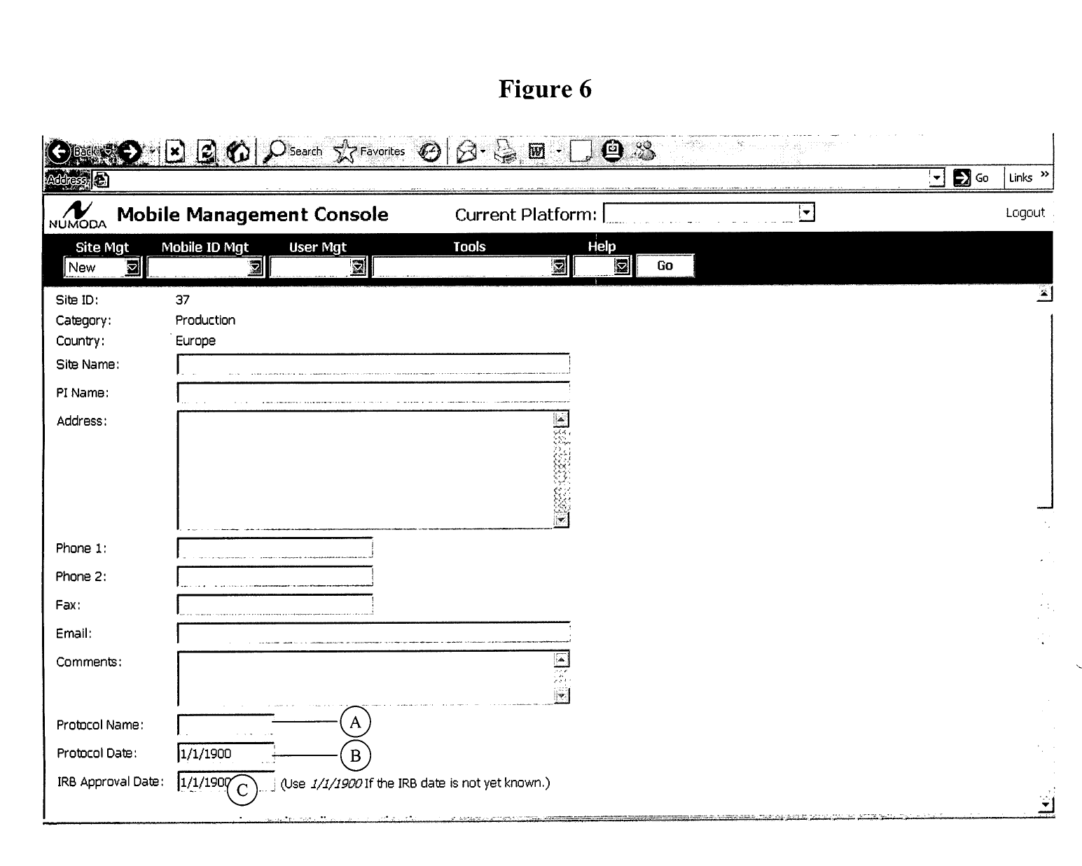

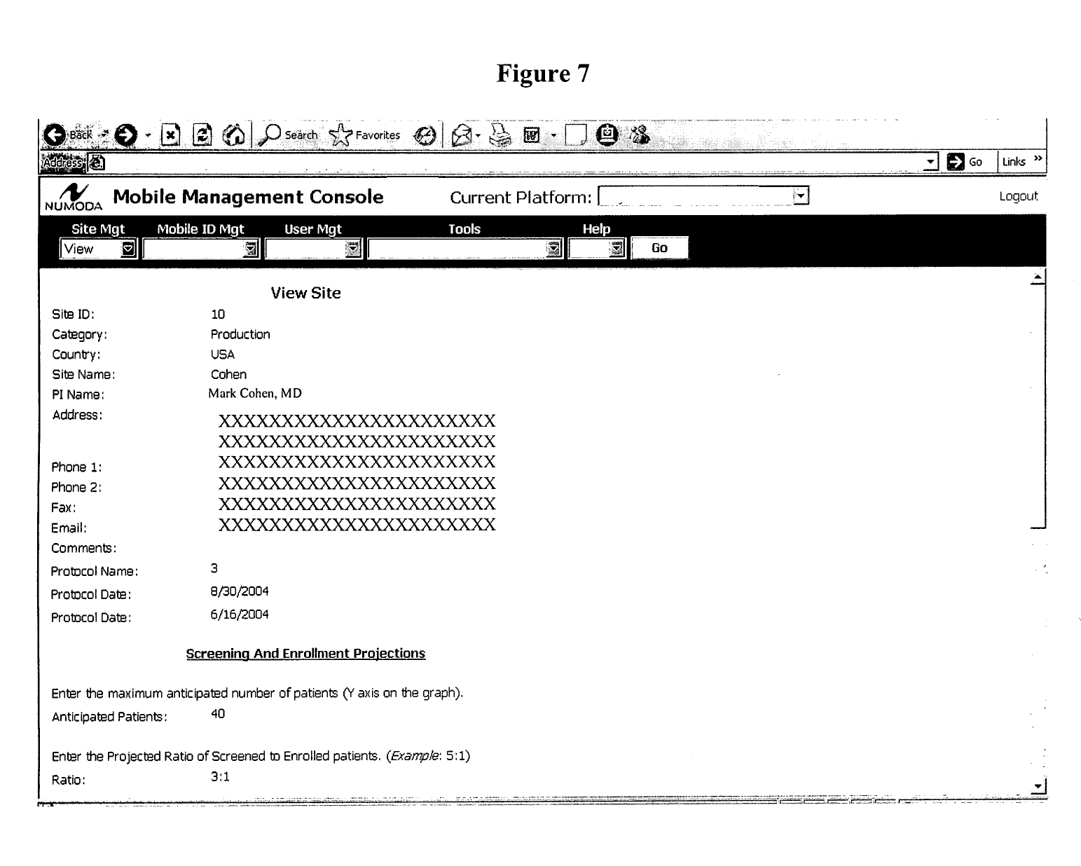

FIGS. 6 and 7 show examples of the algorithms that can be set for the trial for handling protocol, inclusion/exclusion etc versions and how this information can be entered in the system. FIGS. 6 and 7 shows how the present invention enables a site to be added to the system and how algorithms can be set to track screening and enrollment. FIG. 6 shows how a site can be entered into the network, with ‘A’ showing where the name and version of the protocol can be entered for this site. ‘B’ is the field for the date of this version of the protocol, and ‘C’ is the field for the date the protocol was approved. Setting these dates in the system means the system will display the corresponding appropriate protocol versions to the sites. FIG. 7 shows how to view the algorithms that are set.

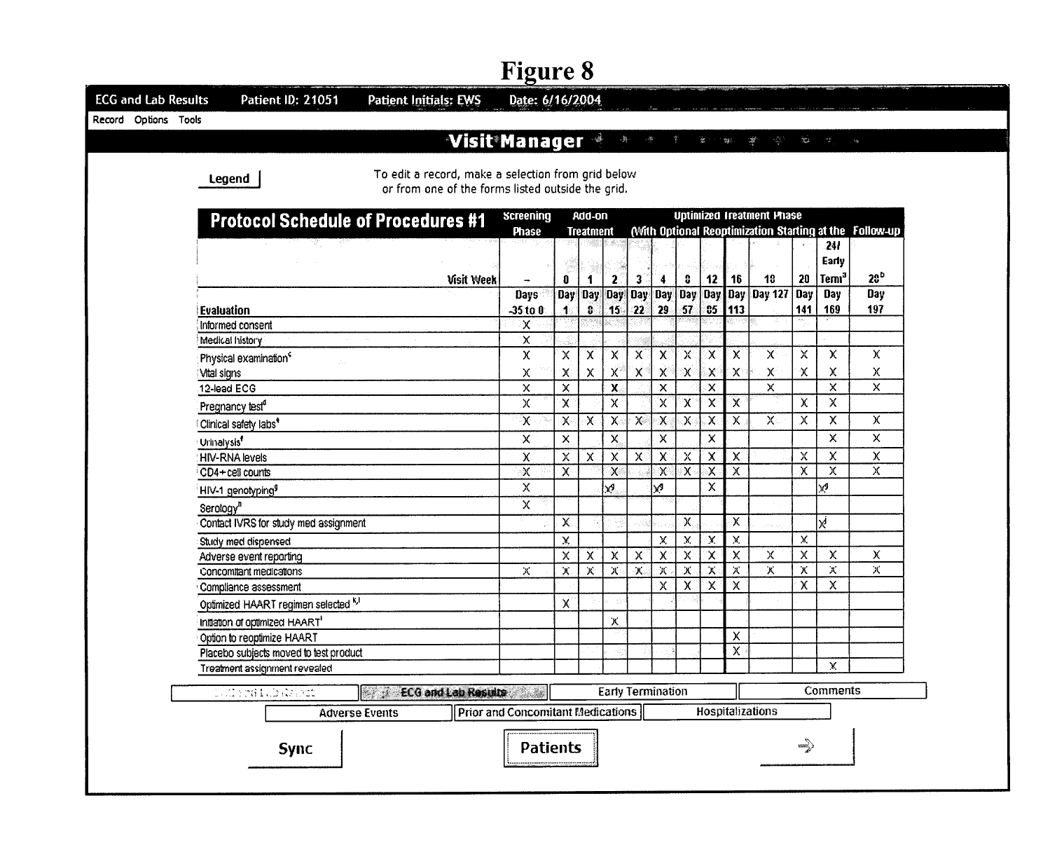

FIG. 8 is a sample screen of the part of the present invention that is deployed at the site. The present invention will display the appropriate procedures for the correct version of the protocol for a site. These boxes for the procedures are only accessible at the appropriate times.

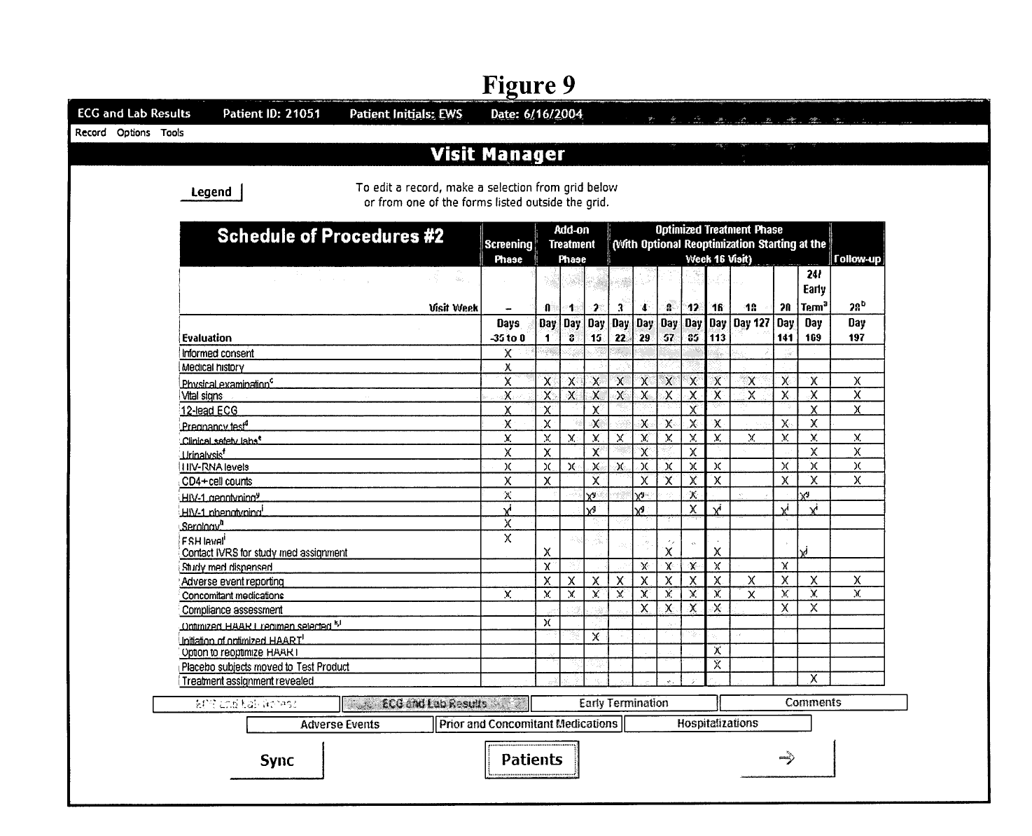

FIG. 9 shows the changes in the protocol that will automatically display for the appropriate site, for the appropriate patient at that site, under the appropriate protocol. This is an important aspect of the present invention since a patient may be originally screened under version one of the protocol, but the protocol has changed during the 35 day screening period and the patient is now being enrolled under version #2 of the protocol. This level of management extends into the individual procedures for the protocol as well.

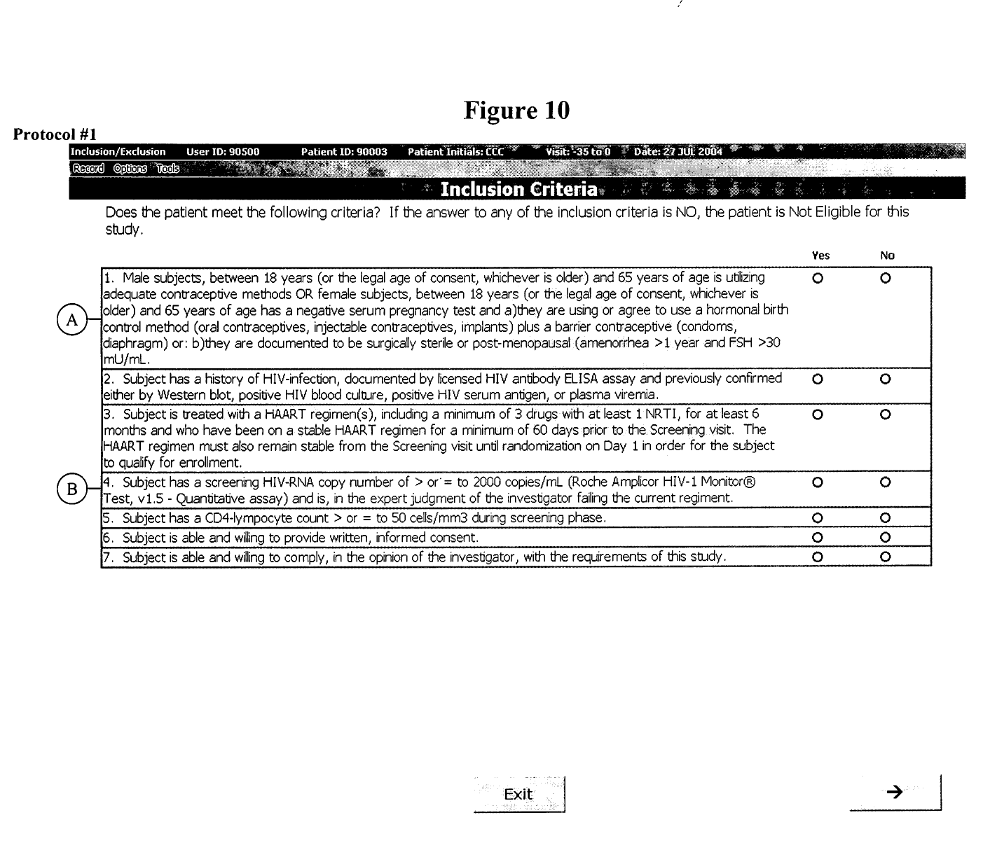

FIG. 10 shows how the site will be given a screen that displays the inclusion criteria for the first version of the protocol. FIG. 10, items ‘A’ and ‘B’ highlight a type of criteria that will be changed. Many other criteria can change as well.

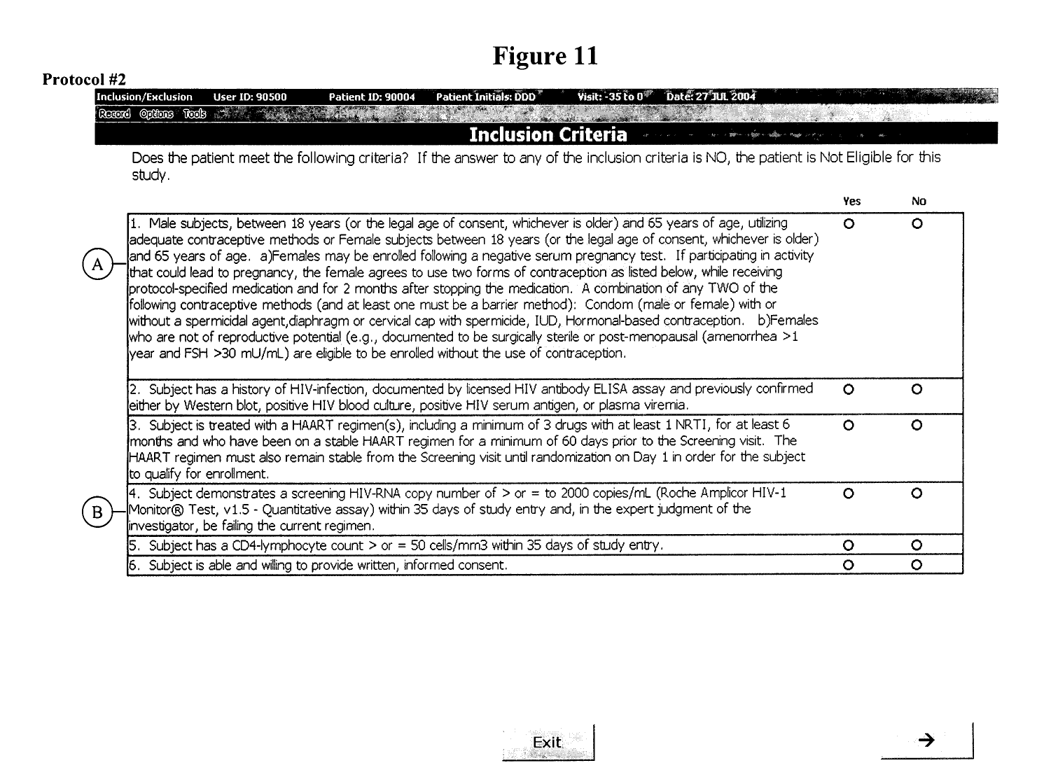

FIG. 11, items ‘A’ and ‘B’ show the screen that the present invention will display, when the site is approved for the new version of the protocol. The present invention can display the correct inclusion/exclusion criteria on a site-by-site, or country-by-country basis.

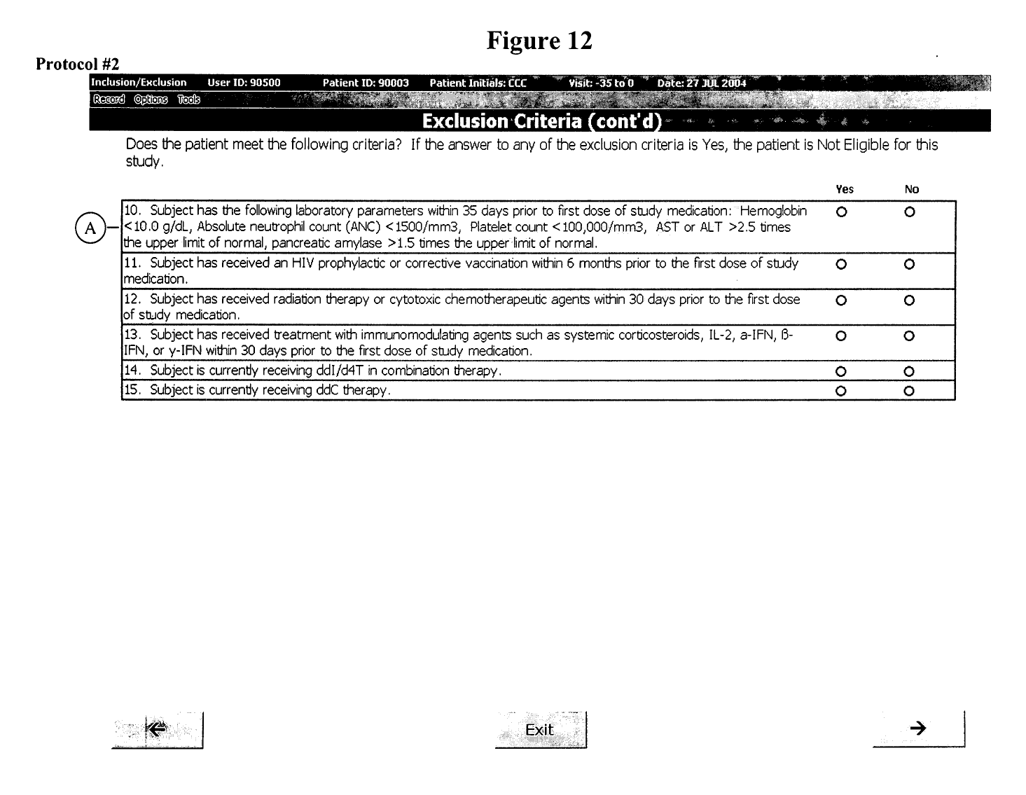

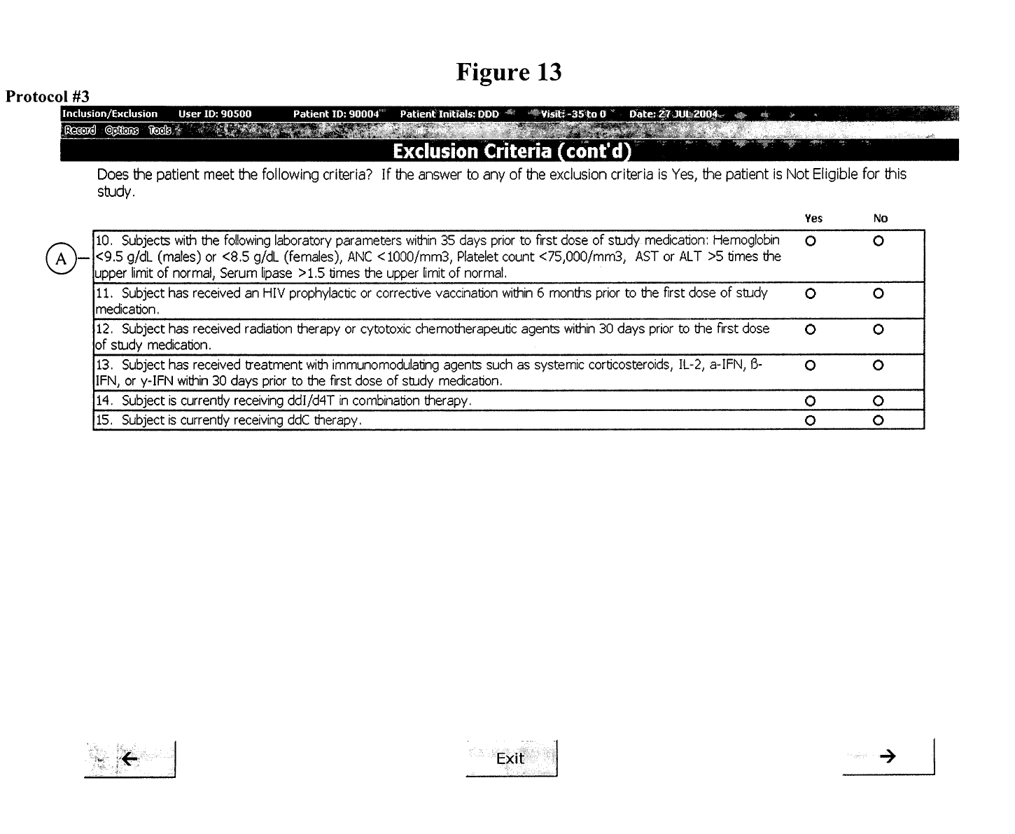

FIG. 12 shows in row ‘A’ an exclusion criteria that is approved in the 2^{nd} version of the protocol. FIG. 13 shows where a different exclusion criteria is displayed to the site, as seen in row ‘A’. With the present invention, the site can simply enter in the date of informed consent and the date of birth and the system will calculate versions of the criteria, policies for waivers, and track the patient to be called back in a certain time period. The present invention will manage and track all these parameters for each patient, resulting in a reduction in errors, a reduction in screen failures and an increase in enrollment. An additional aspect of the present invention will report this information to the study team and calculate that with a change in policy to calculate the age by the date of randomization rather than the date of informed consent, a higher percentage of patients would be enrolled. Further error and logistical complexity can be reduced as the present invention provides more complex calculations and manages more versions of inclusion/exclusion criteria.

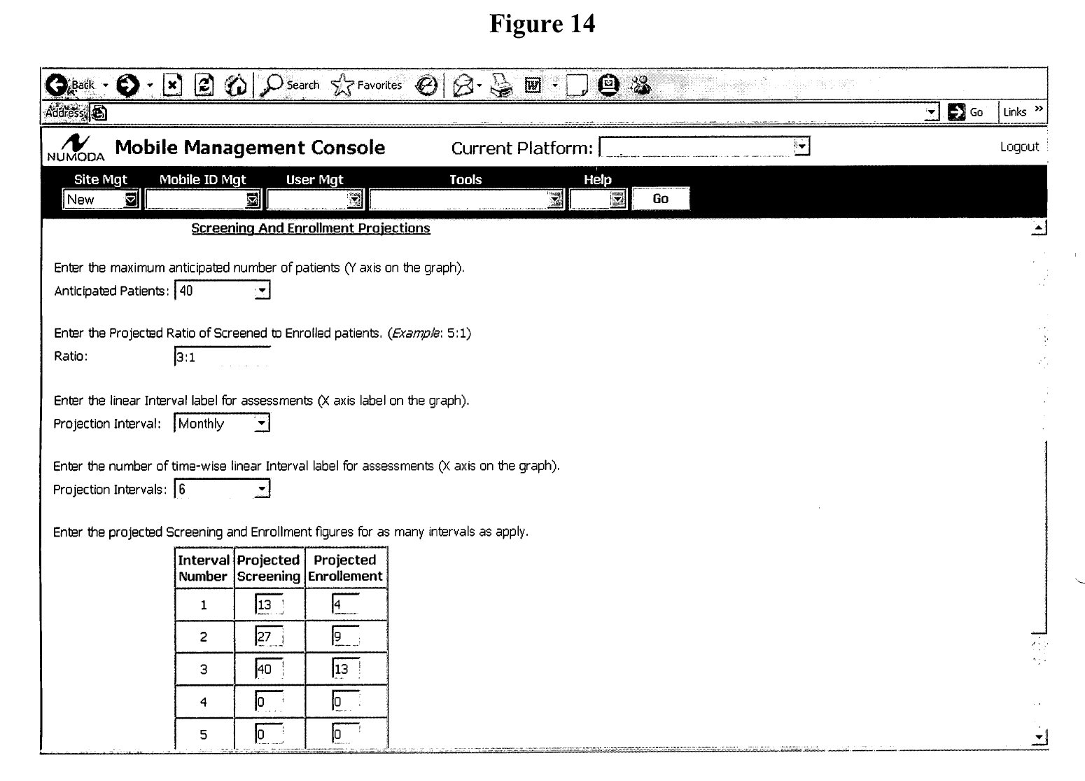

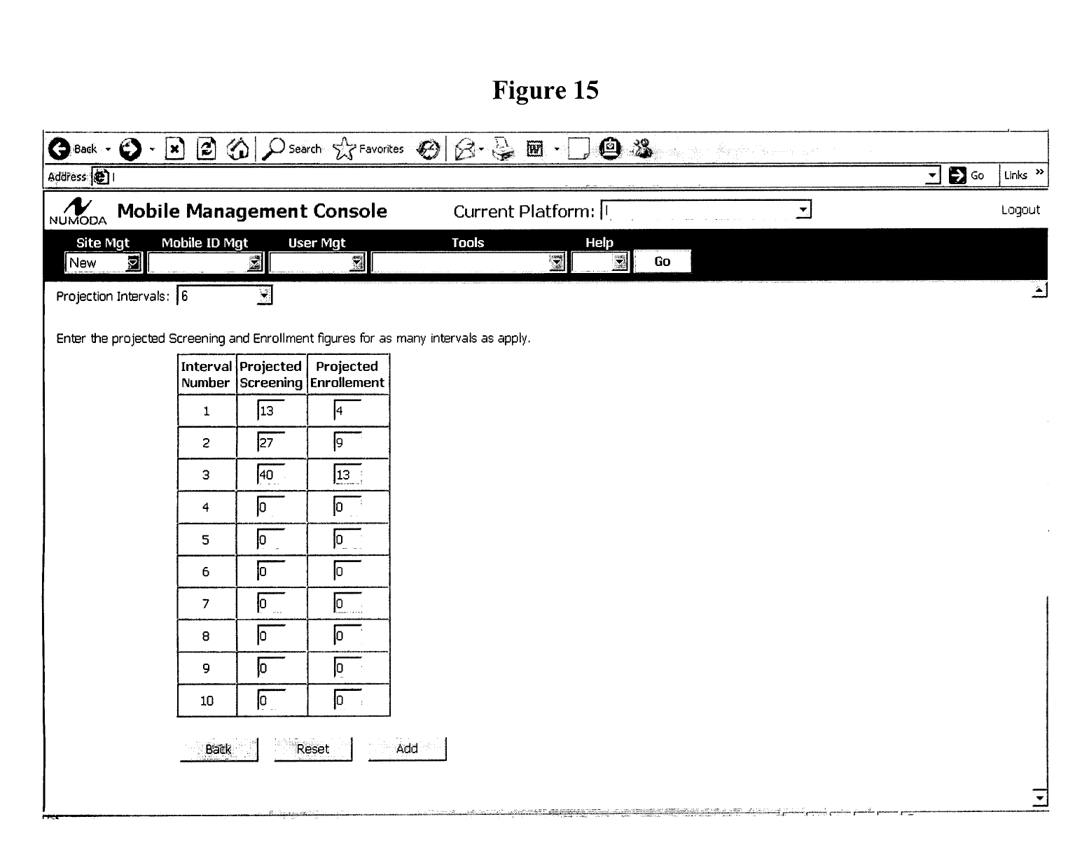

FIGS. 14 and 15 show examples of the algorithms that can be set for managing the projected vs. actual enrollment.

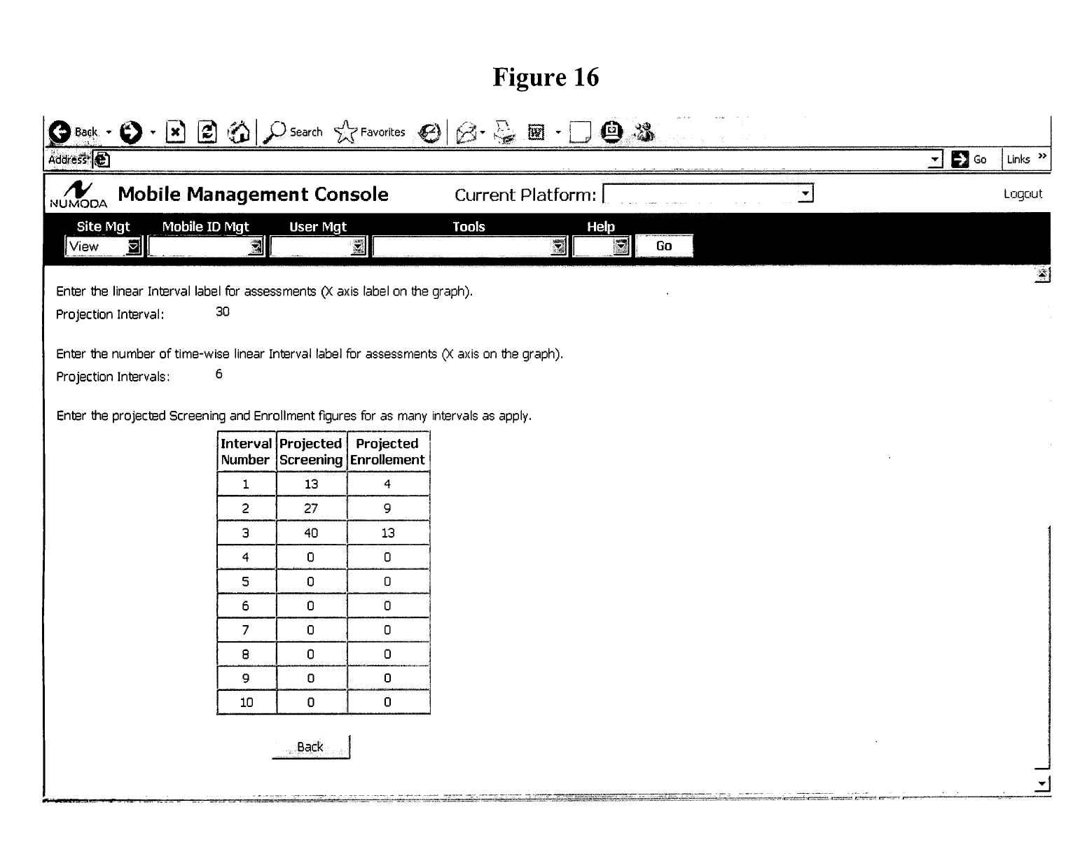

FIG. 16 shows the algorithms that are set. FIG. 16 also shows the fields for setting the projected enrollment. Setting these numbers will allow the system to compare with the actual enrollment being obtained in the system and then the system automatically calculates and projects whether enrollment is on target with expectations. Further projections are made by the system to establish the need for an additional number of sites to be added, or identify sites that don't have patients that can be evaluated, in order to focus on sites that do reach a sufficient number of enrolled patients.

The investigator sites are automatically notified by the present invention, regarding reports that pertain to the sites' activity regarding screening and enrolling patients. These reports, forwarded to the sites by the present invention, show the staff at the site precisely how many patients they are enrolling compared to their estimated or contracted performance. Sites may also receive notification of their performance compared to other sites. This information is always useful and eliminates the need for the sites to perform these calculations and provide sites with accurate and timely payments for enrollment. It is also important when the enrollment for the study is on a competitive basis. Sites will immediately be aware that it is no longer necessary to screen additional patients when the correct number of patients is enrolled. Without the present invention, sites are often not aware that the enrollment was reached for the study and the site continues the expensive work of screening more patients when it is no longer necessary.

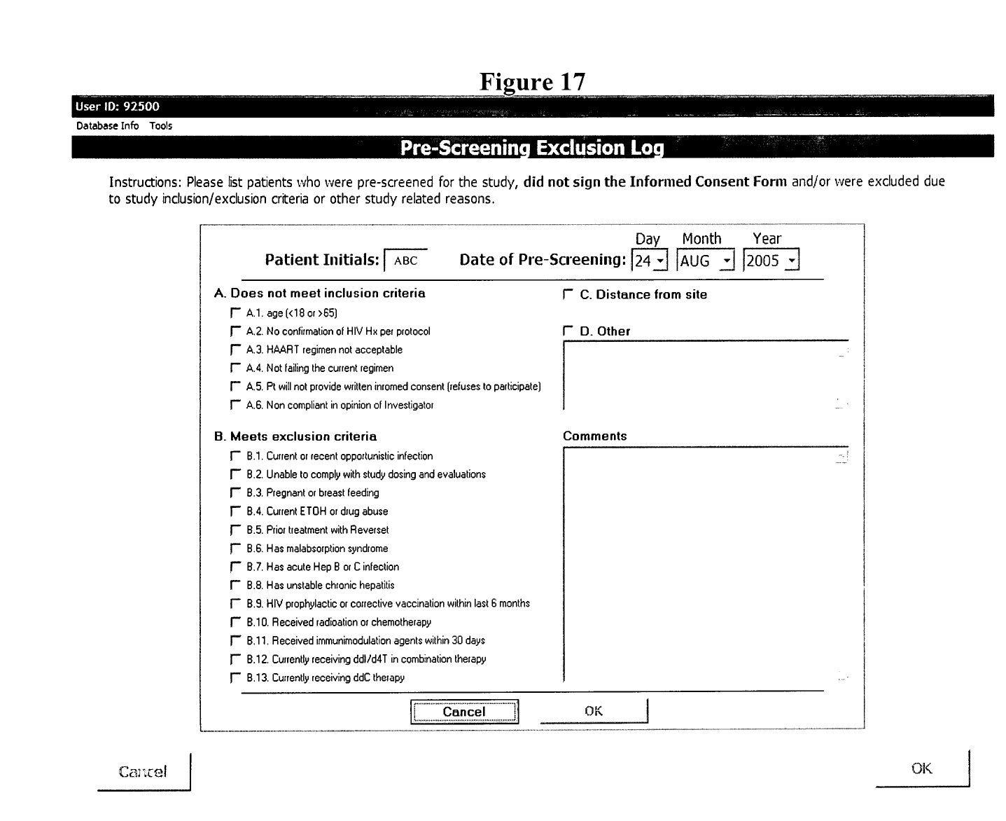

FIG. 17 shows how the site can simply select from preconfigured choices, or can add others. This makes it simple for the site to record and report at the same time, the reason that the patient did not sign the consent, e.g. cannot afford bus fare to the site. With this information immediately recorded and reported, the study team can make a more informed decision about changing the policy to allow for the payment of bus fare. Rather than sporadic reporting, the study team now knows how many patients have refused or were ineligible to be part of the study and for what reason(s).

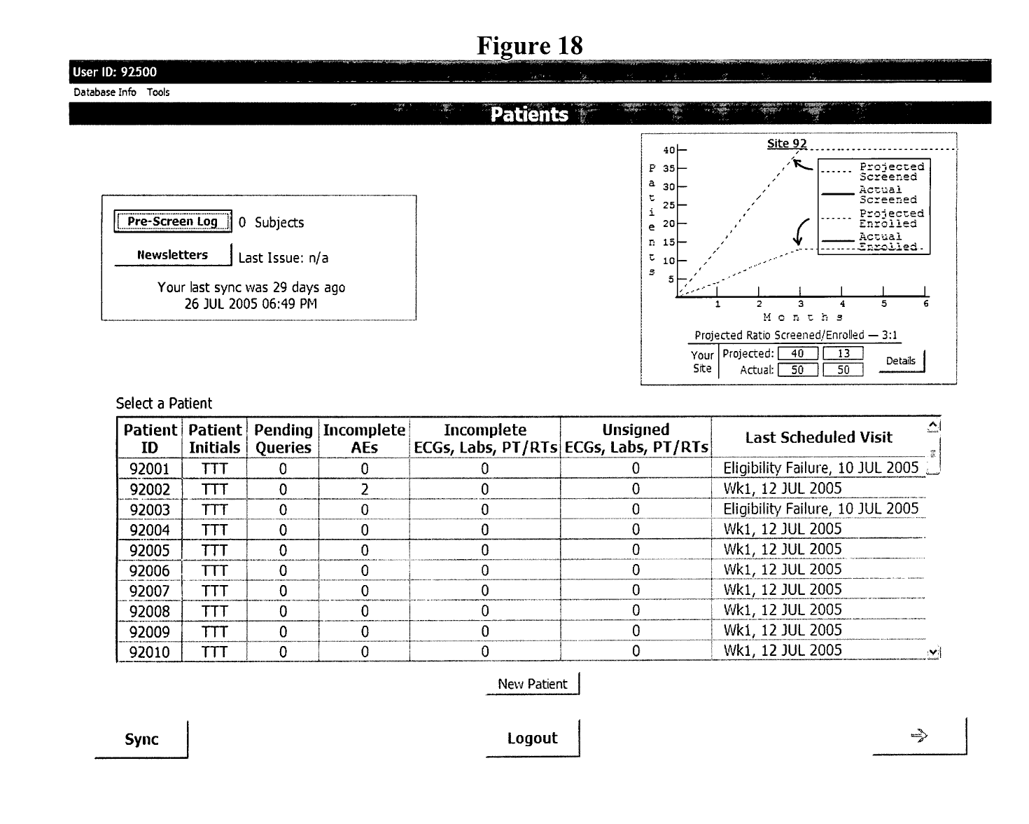

FIG. 18 shows how the present invention will display to the sites, a console that summarizes the information on projected and actual enrolment and shows appropriate details on patients that are eligibility failures (failed screening). Any other information that is necessary for screening and enrollment can be displayed on these screens as well. The present invention also provides diagnostic tools for the sites, that calculate laboratory values, against accepted clinical norms and display information that a patient would be appropriate for the study.

Additional tools are used to set up the integrations of the numerous other systems that will provide data that will be processed by the present invention.

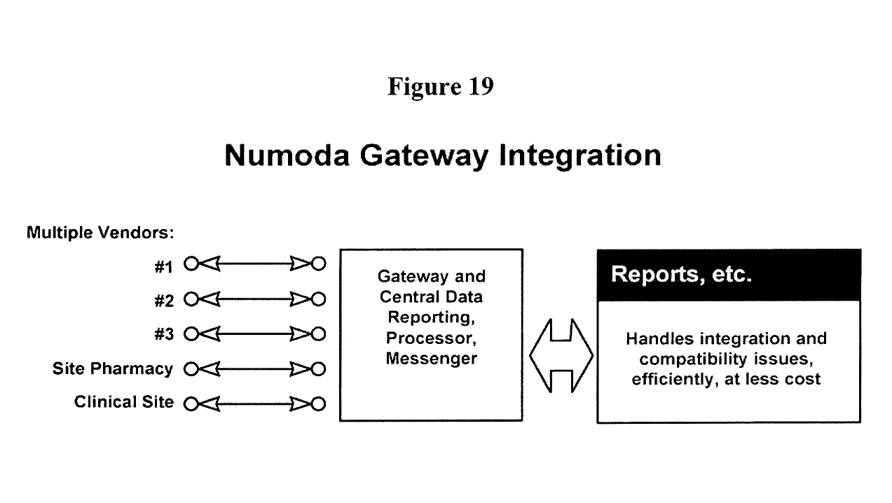

FIG. 19 depicts how the system acts as a gateway that draws in the necessary information from any number of disparate systems that are part of the screening and enrollment process. (FIG. 5 ‘F’ also shows this gateway.) This data is drawn into a single unified system. There is only a single point of integration that needs to be updated when any changes occur. Data is processed within the system. Compatibility is secured, upgrades can be accommodated on both sides, quickly, and at less cost, and there is no limit on the options of vendors that can be selected. This makes consolidating and processing the information into reports, easier (and lab results are part of inclusion/exclusion criteria) and provided in real-time. This configuration eliminates the burden of getting reports from many disparate systems and then manually pulling together the information needed to get a report on the most current status of enrollment or on the reasons that enrollment is not going as projected.

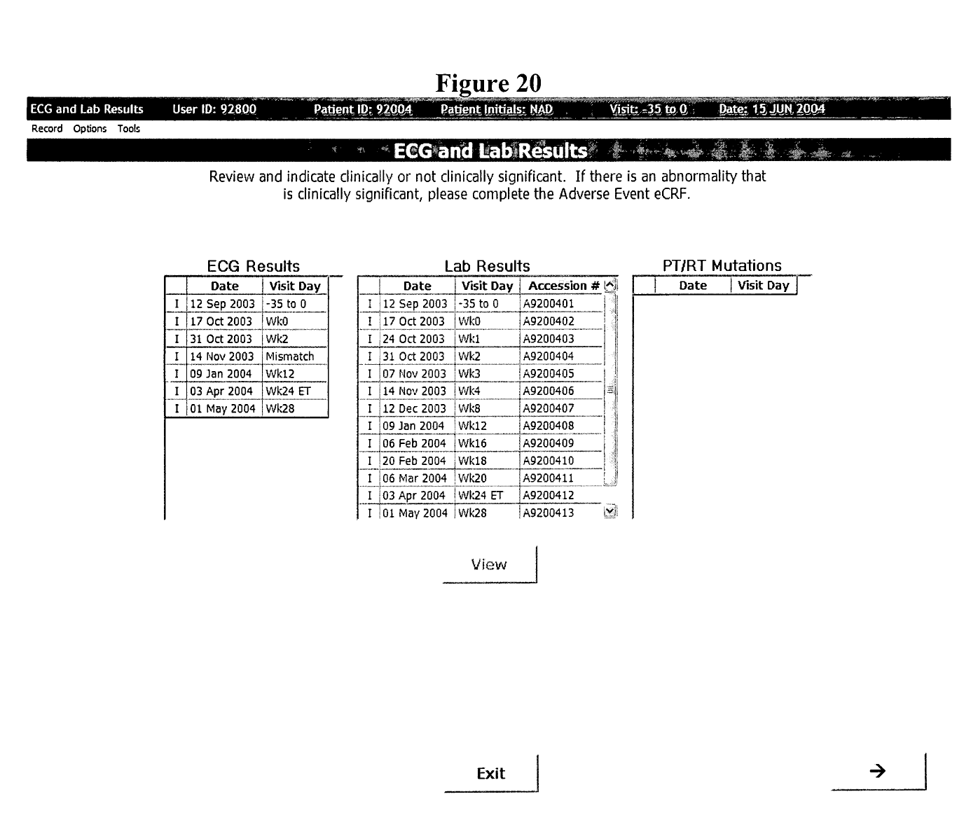

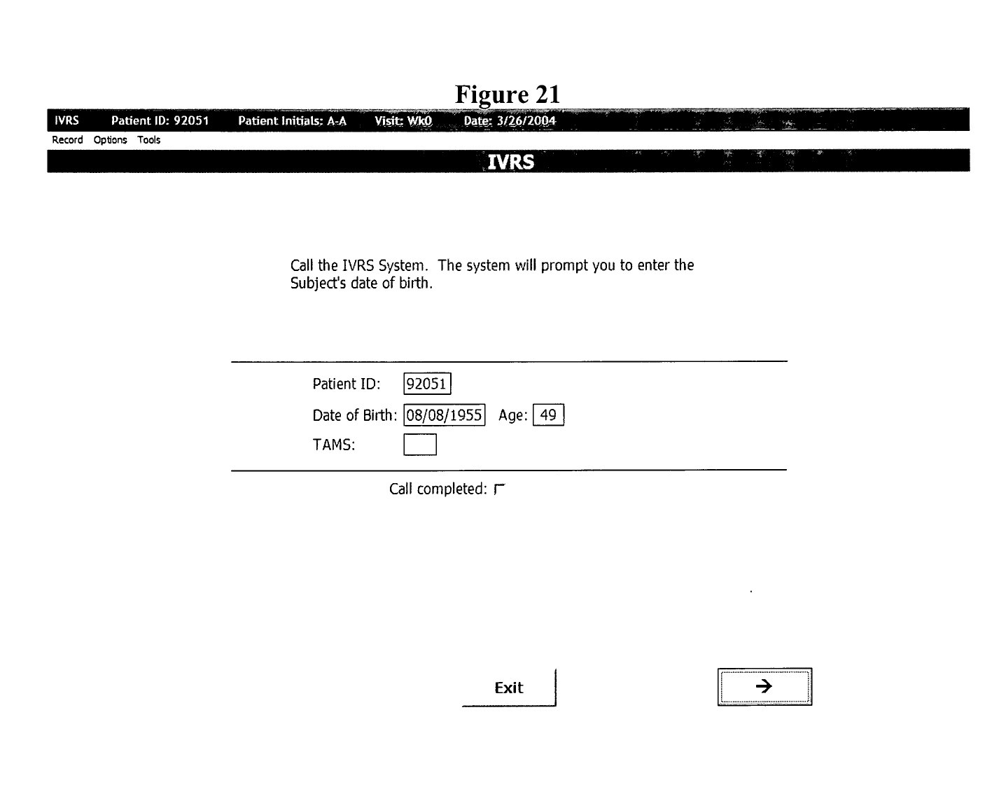

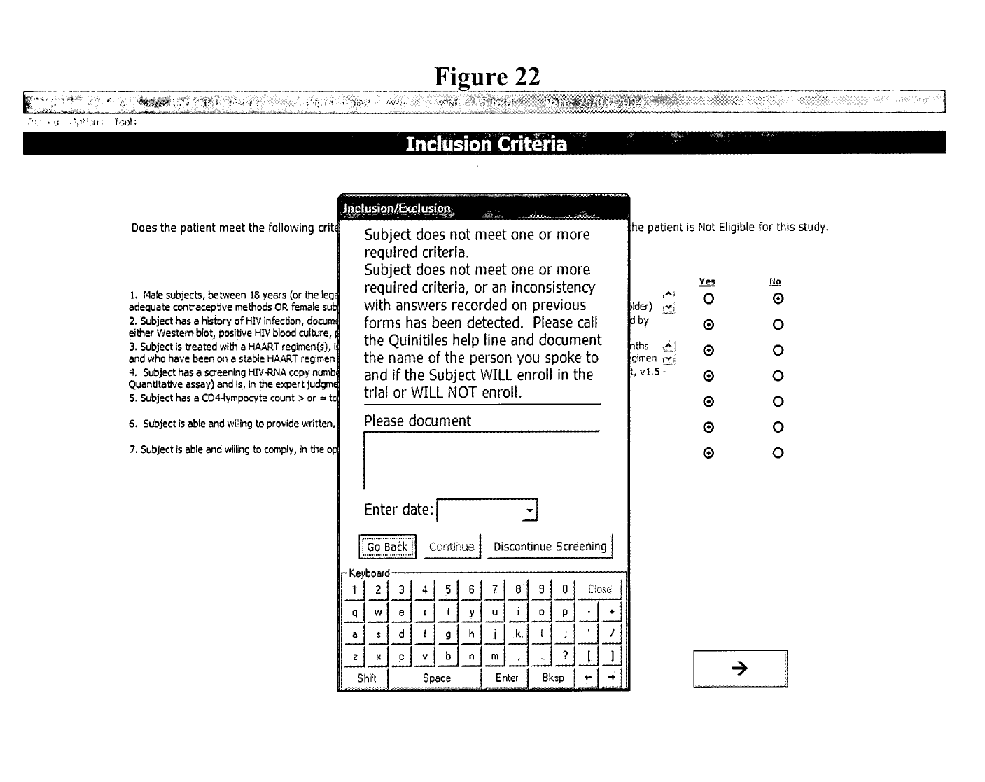

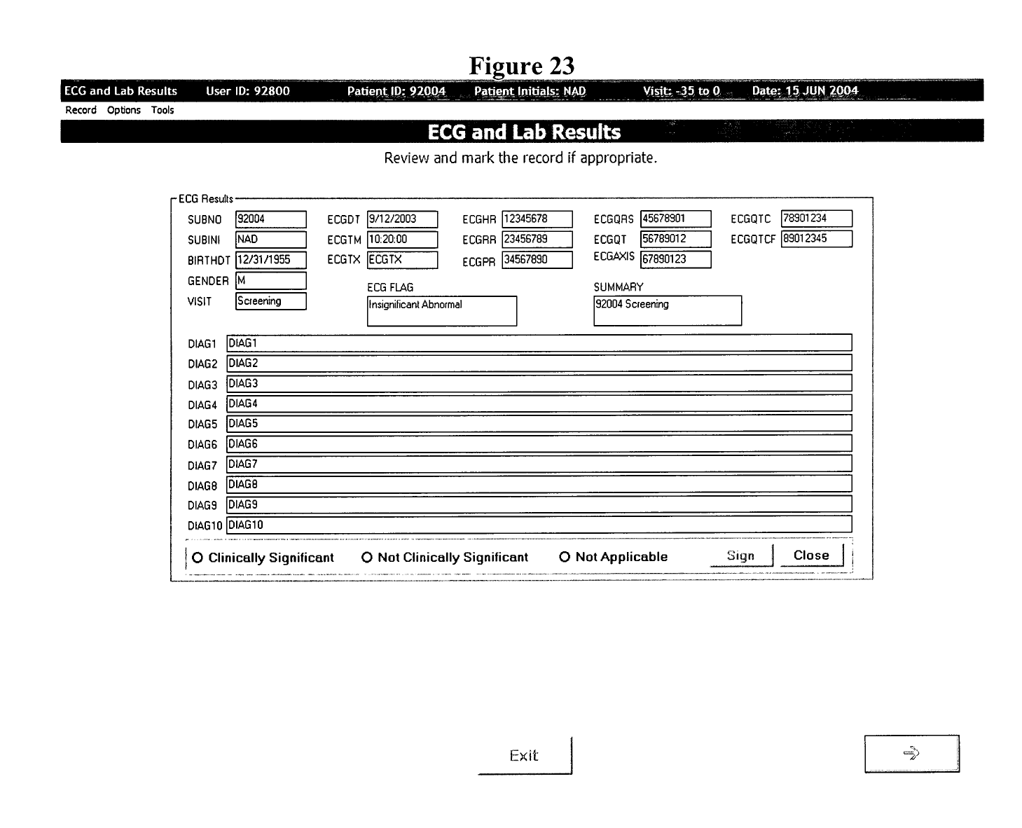

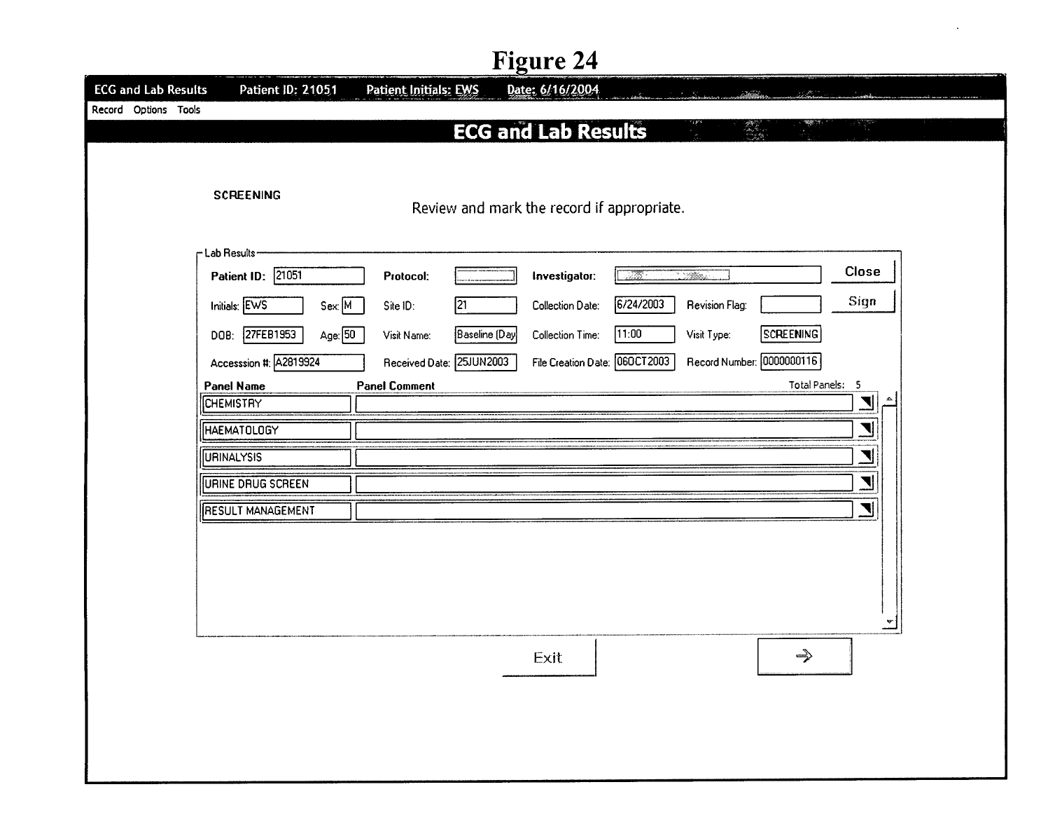

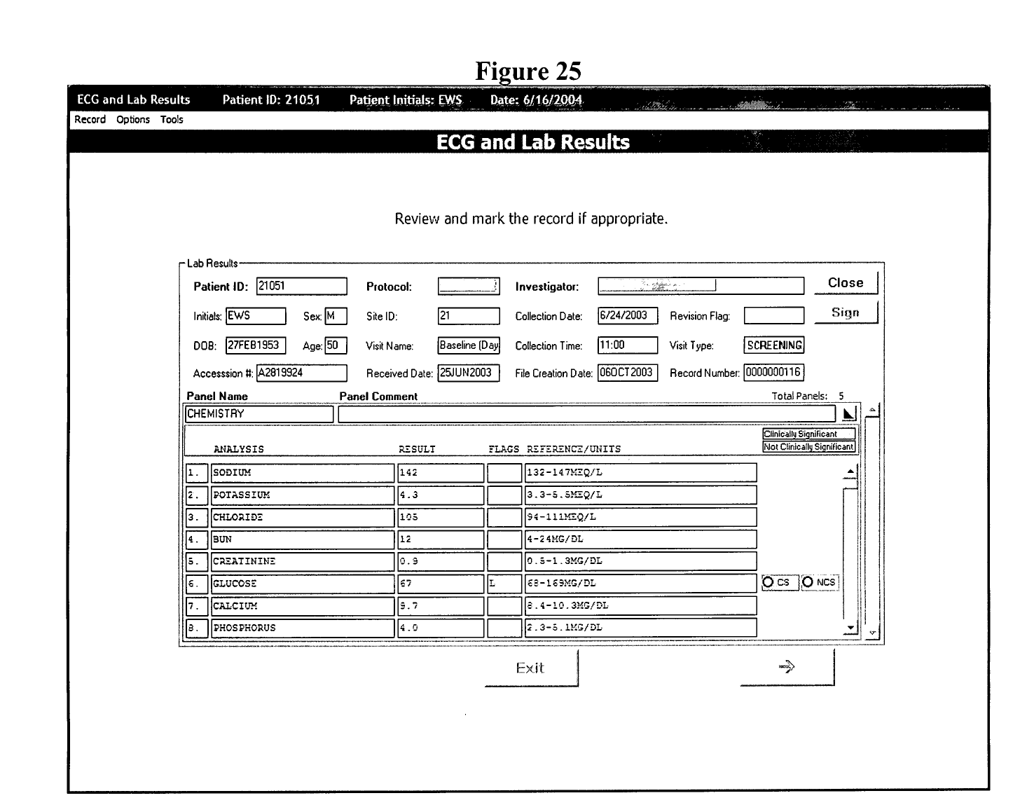

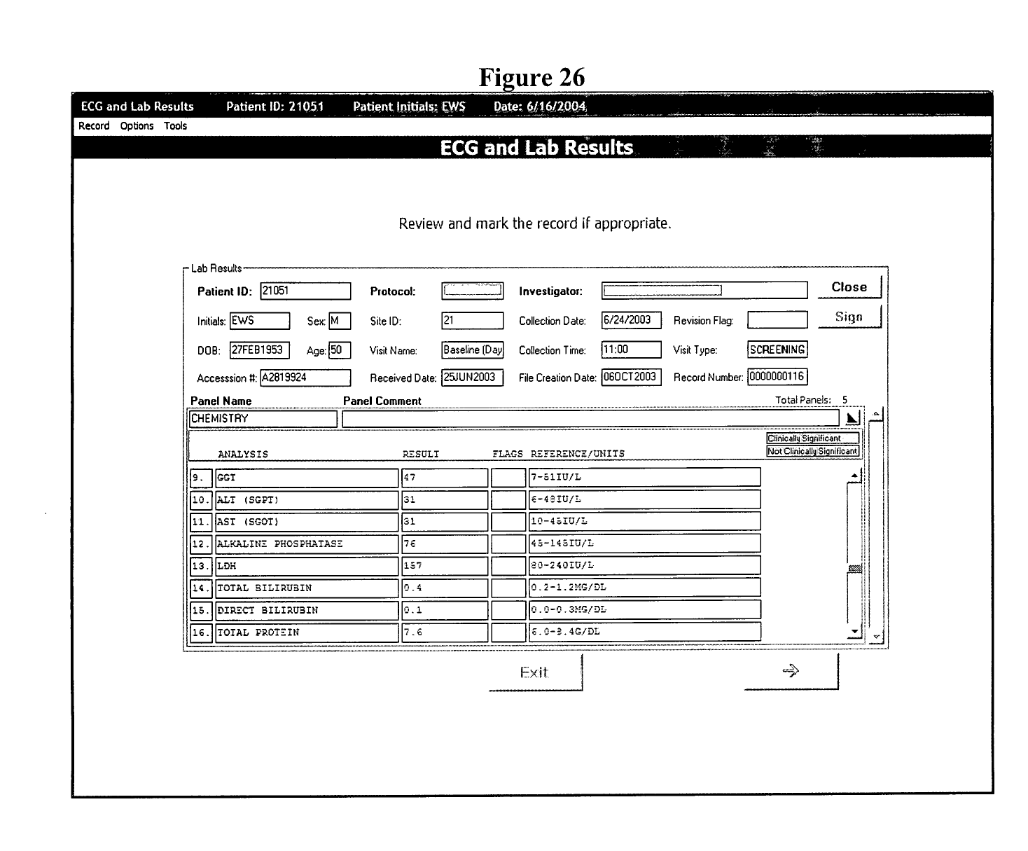

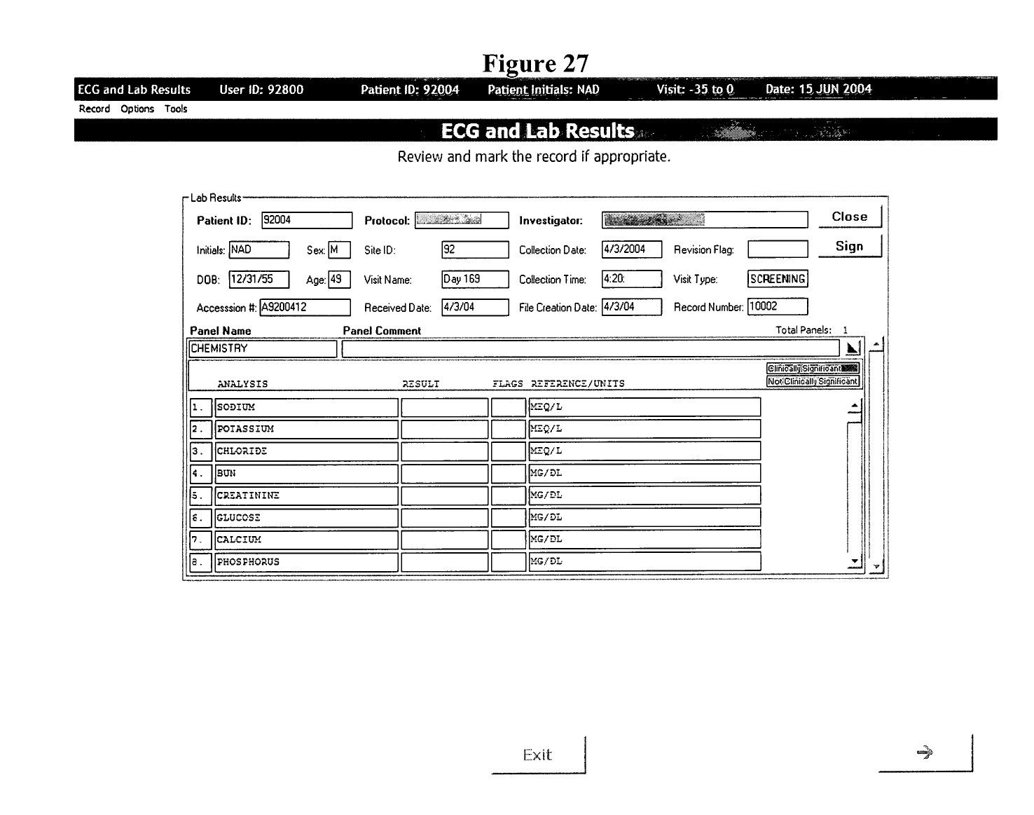

FIGS. 20-27 are screens of how the present invention displays the integrated reports of lab, ECG, and specialty labs (PT/RT) on the tools at the investigator sites. FIG. 20 shows all of the lab reports displayed and information about the lab reports, such as whether a particular lab was part of the screening visit (Visit Day—35 to 0), or part of the enrollment visit (Visit Day Wk0). Any of these can be selected and viewed. The display and access of this information for the sites eliminates the need for the sites to gather this information from multiple sources. This means that patients are more accurately and more quickly enrolled into the trial. In addition, with this diagnostic result information managed and consolidated by the present invention, the present invention will compare and calculate these values against the multiple versions of the inclusion/exclusion criteria for the trial. FIG. 21 is an example of how the present invention will message the site with the results of screening labs. FIG. 22 is an example of how the present invention will consolidate this screening lab for genotyping with the algorithm for stratification. The genotyping result is a 10-page detail on RNA mutations that is performed and then provided by a specialty lab vendor. Very few people are trained to read this lab results and many mistakes are made in calculating the number of TAMS from this lab result. The present invention will receive the results through the gateway, calculate the number of TAMS based on the appropriate programmed algorithm and once consolidated and calculated, the present invention messages the TAMS number for each patient to the site where the patient will visit. FIG. 21 is an example of how the present invention will message the site with the TAMS number. FIG. 22 is an example of how the present invention will display a prompt for a site member to call to speak with someone about a waiver to include this patient in the trial. For example, this is done if the patient's lab results are outside the accepted value under protocol one but will be accepted under protocol two but this site has not yet received approval for protocol two. FIG. 23 is an example of how the present invention will message the site with screening ECGs. FIG. 24 is an example of how the present invention will message the site with the results of the screening labs. FIGS. 25 and 26, taken together, is an example of the detailed lab results that the present invention will provide. There is no limit to the messages and details that the present invention can provide. FIG. 27 is an example of how the present invention will message another type of labs. All of the recorded information are validated by the present invention and have an audit trial. The present invention accommodates any disparate systems through the gateway and any external supplier can be removed from the system and a new supplier connected through the gateway.

The present invention also makes processed data instantly available and easily accessible through a web-based interface for reporting and access to record additional data related to the trial. This reportal gives access to the people who will be making changes to the trial e.g. new protocol versions, and will give access to the people who will be adding new information to the trial data. Others will simply use the reportal to deliver messages, and receive or access reports. There is no need for the study team to make calls to sites for any of the information that is available in the reportal. Sites do not need to be interrupted during their busy schedule, to answer questions for the sponsor, or fax and mail data to the sponsor. Sites have records of all their information as part of their daily work at the site, using the present invention, and the present invention automatically consolidates all data from any source that is part of the screening process such as lab vendors, ECG processing companies, and specialty labs, clinical materials suppliers, etc., and makes this part of the site record as well. The present invention's central repository organizes this information in real time, and the present invention provides analysis and trending information to the study team. This information, in the form of reports from the present invention, will help the study team make better decisions. For example, the present invention gives the appropriate person the information that patients need payment for bus fare to enroll in the trial. This person can then decide on a policy to reimburse for transportation expenses or notify specific sites that payment for transportation is a current policy. This information is available without the need for data collection and follow-on data entry, making notification regarding this issue, a rapid turn around time. Information such as the rate of success of a specific recruitment plan, the value vs. cost of a change in protocol e.g. to accept abstinence as a form of birth control, etc. lets the study team know the specific impact that these changes will make, on the enrollment for the study.

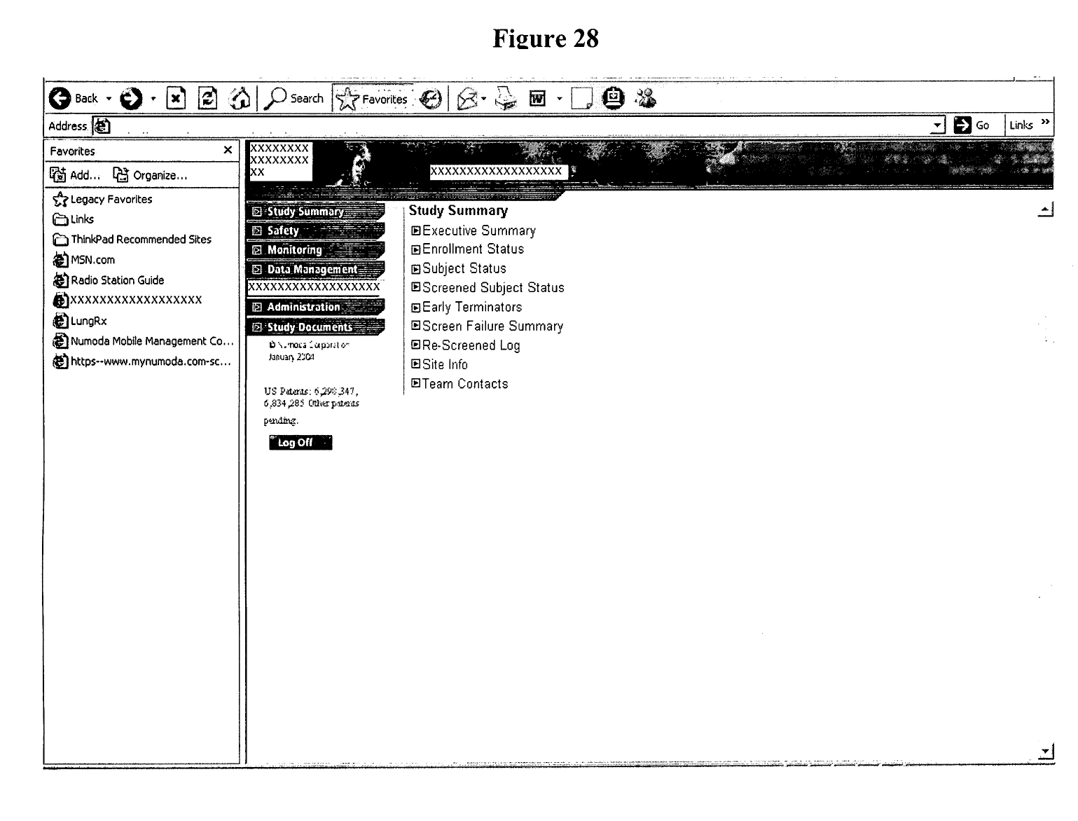

FIGS. 28-47 are examples of reports that are generated by the present invention and accessed through the reportal. These reports are directly available to the study team, through a web based reportal or messages are automatically sent directly to the person responsible to act on the information. All reports are updated immediately, when new data is available in the system. The web reportal is available to the study team to access through their computer, through the internet, any time of the day, from anywhere in the world. These web reports that are generated live, without programmer intervention.

FIG. 28 is an example of the screening and enrollment reports that are generated by the present invention. There is no limit to the type of reports since, all of the information for the study is available for reporting.

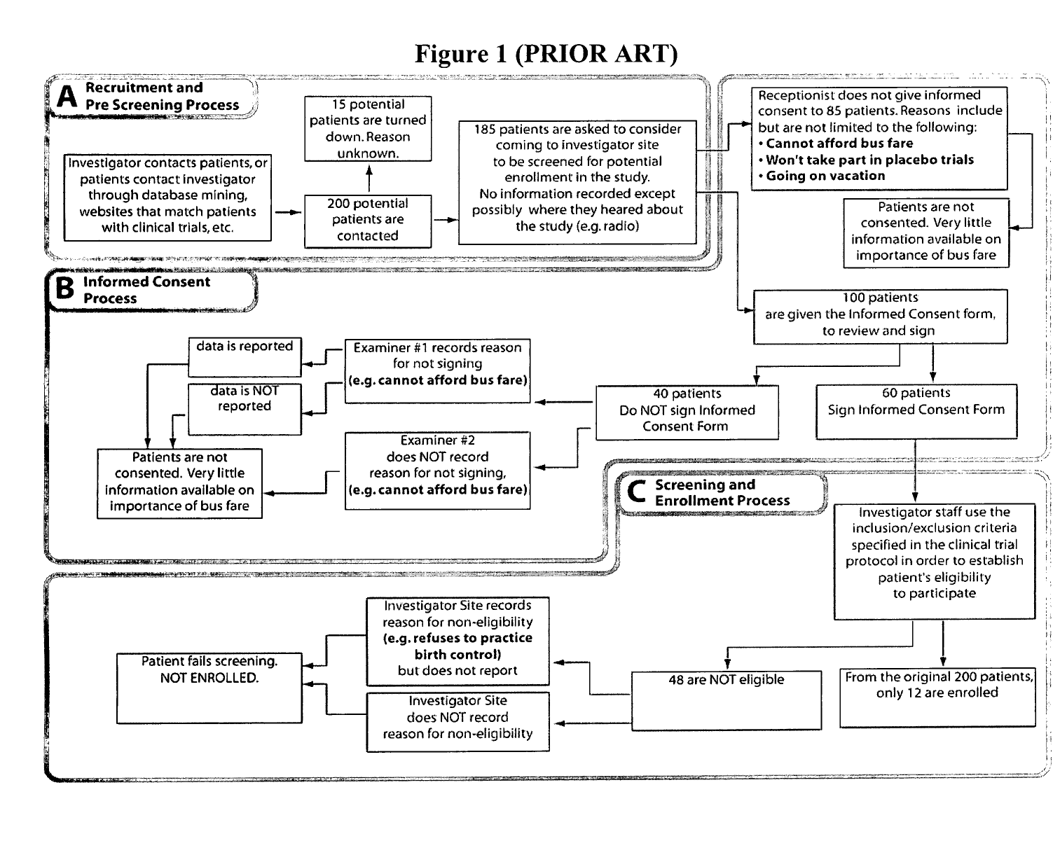

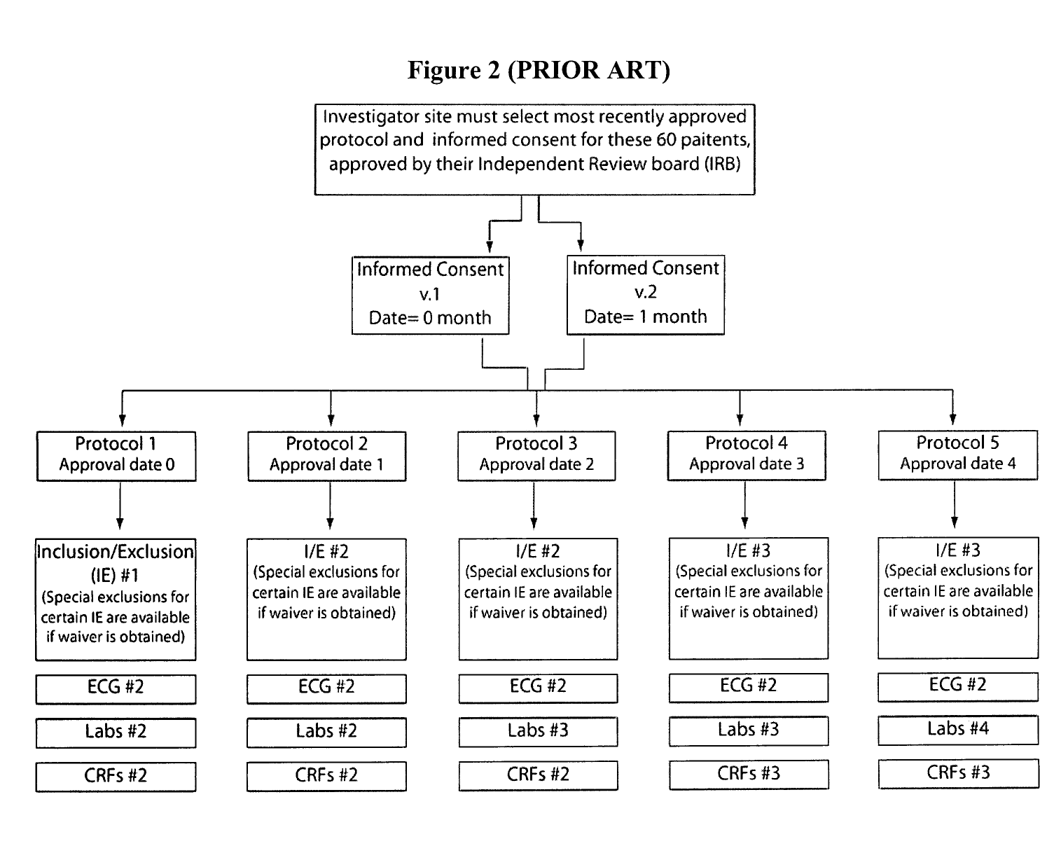

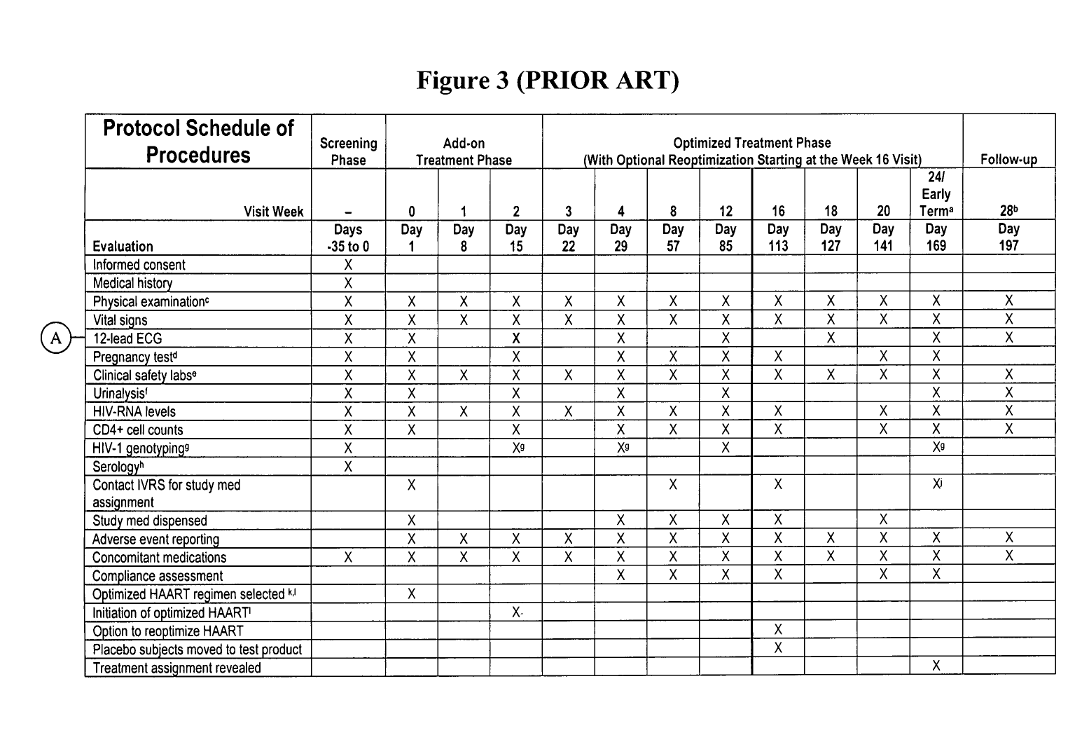

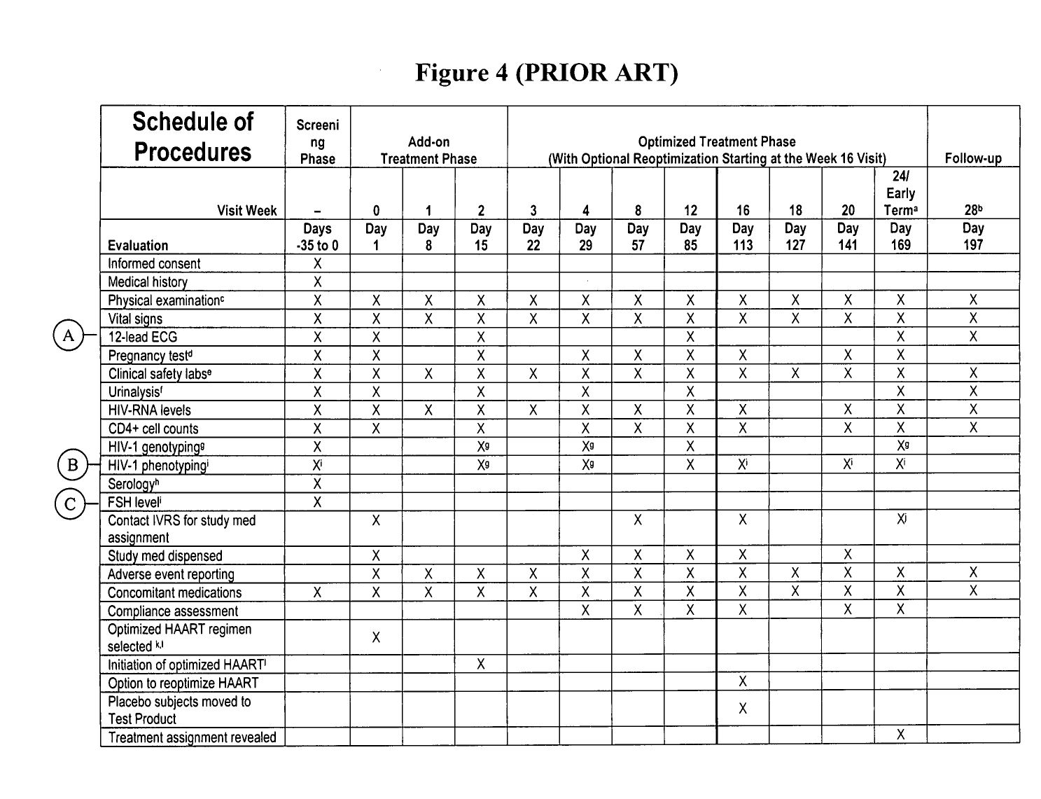

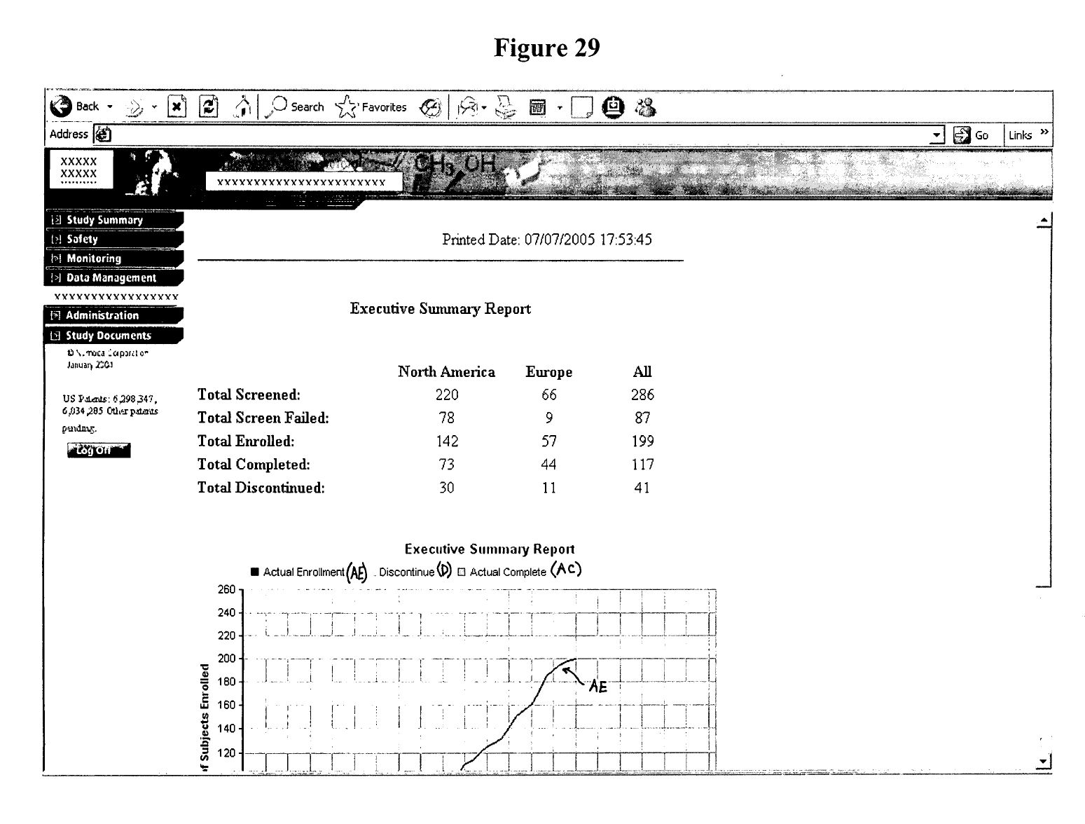

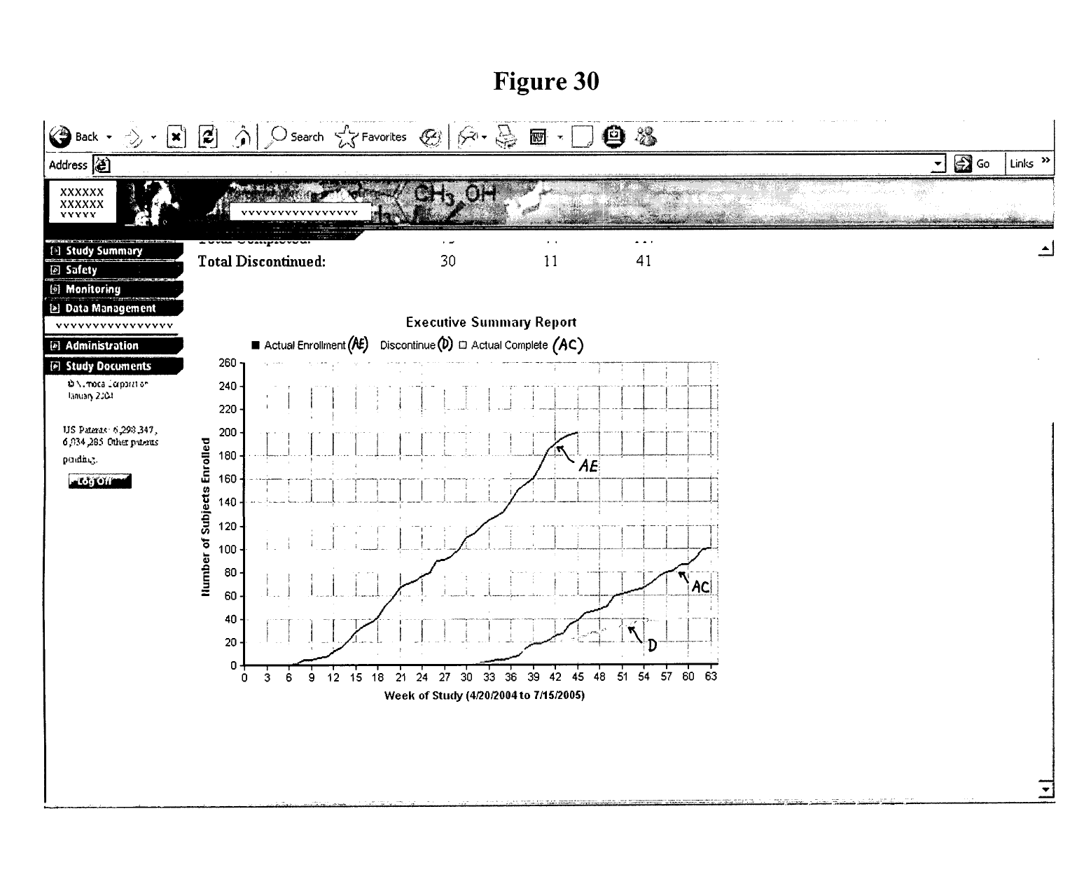

FIGS. 29 and 30 show an example of how the present invention will generate a graphic and the actual numbers for the status of patients that are going through the process of informed consent and screening and enrollment as has been discussed in FIG. 1, steps B and C. Also included are patients that have completed the study and patients who were enrolled but discontinued in the study. All the information is summarized by country and is presented as totals. The present invention has consolidated the information from every site and every country. It has compared where duplicate information has come into the central repository and has accounted for the duplicates and not shown them in the report. The present invention did not require and data entry or manual intervention to show this report. It has consolidated the information in separate databases, and processed it accurately to be shown in this report.

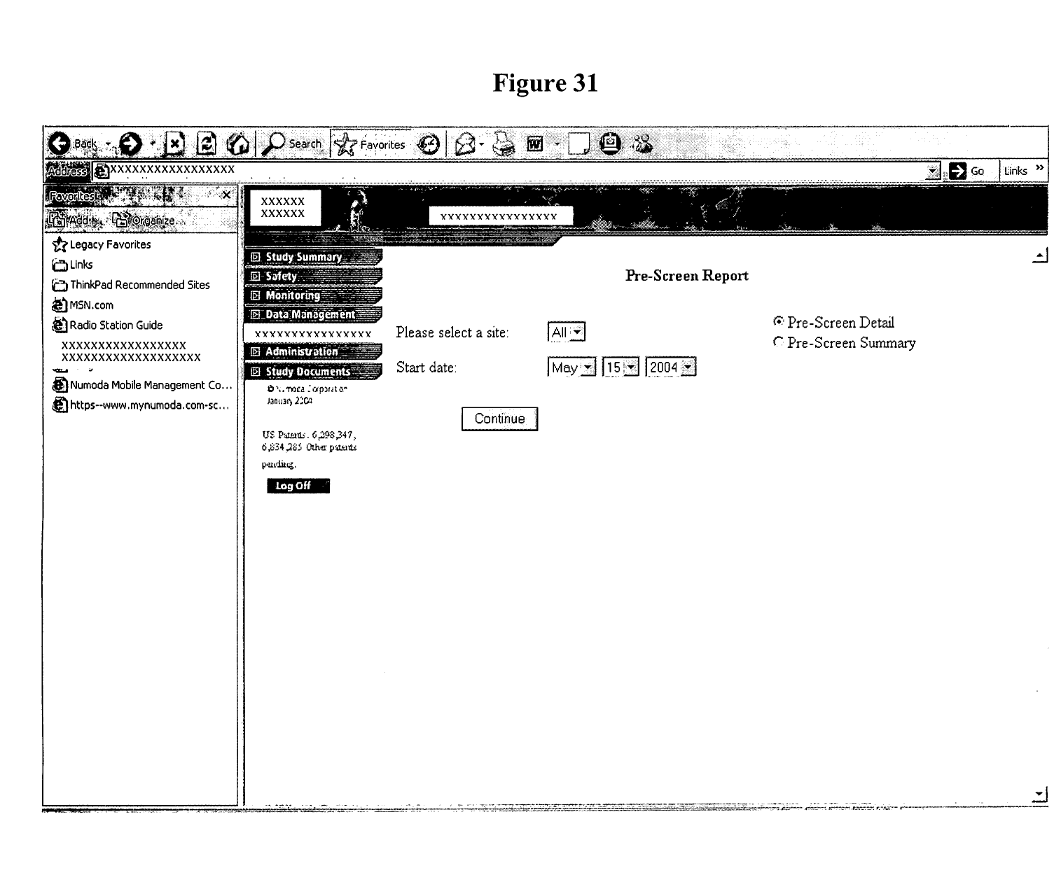

FIG. 31 shows how the present invention will enable a member of the study team to generate a prescreen report for a number of parameters such as site, date, etc. The team member can view information on why patients were not given the informed consent or did not sign informed consent when it was given to them.

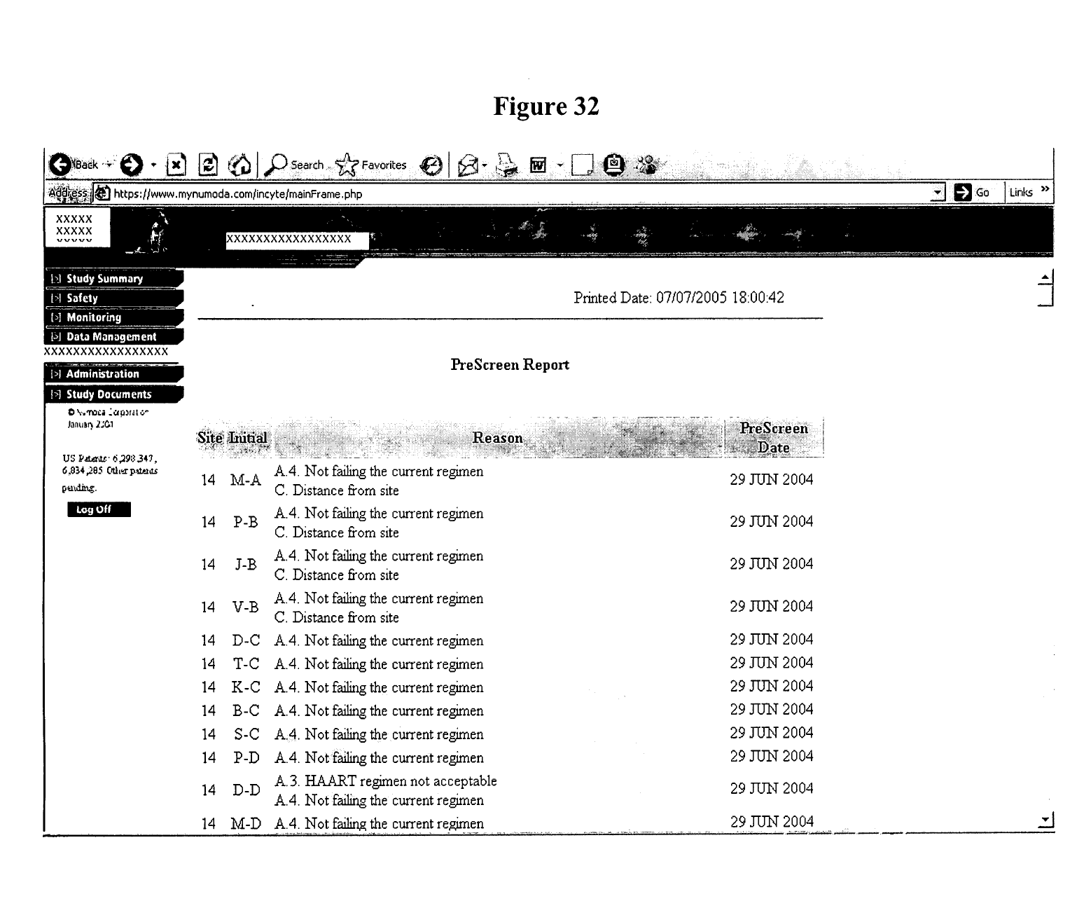

FIG. 32 is an example of a report from the present invention. This report contains details such as the site number, the reason for not entering the study, date, etc. This information is also available in reports that analyze and trend this information across sites. The study team uses these the information provided by these reports, to proactively plan interventions at the site level for example, additional training on accepted practices for the study. The study team can also plan for global interventions at all sites, for example by initiating a new version of the study protocol.

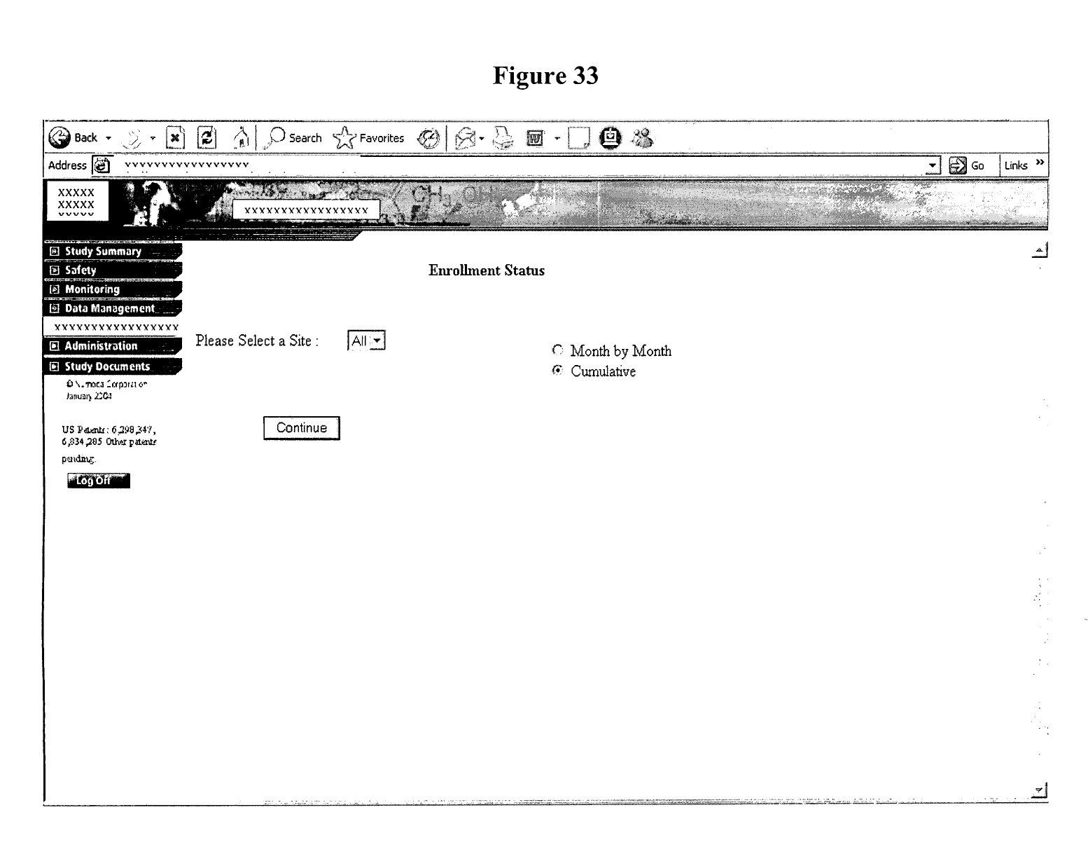

FIG. 33 is an example of how the present invention can provide the information in a number of ways—either by site, month-by-month, or cumulative. There is no limitation as to the reports that the present invention can provide.

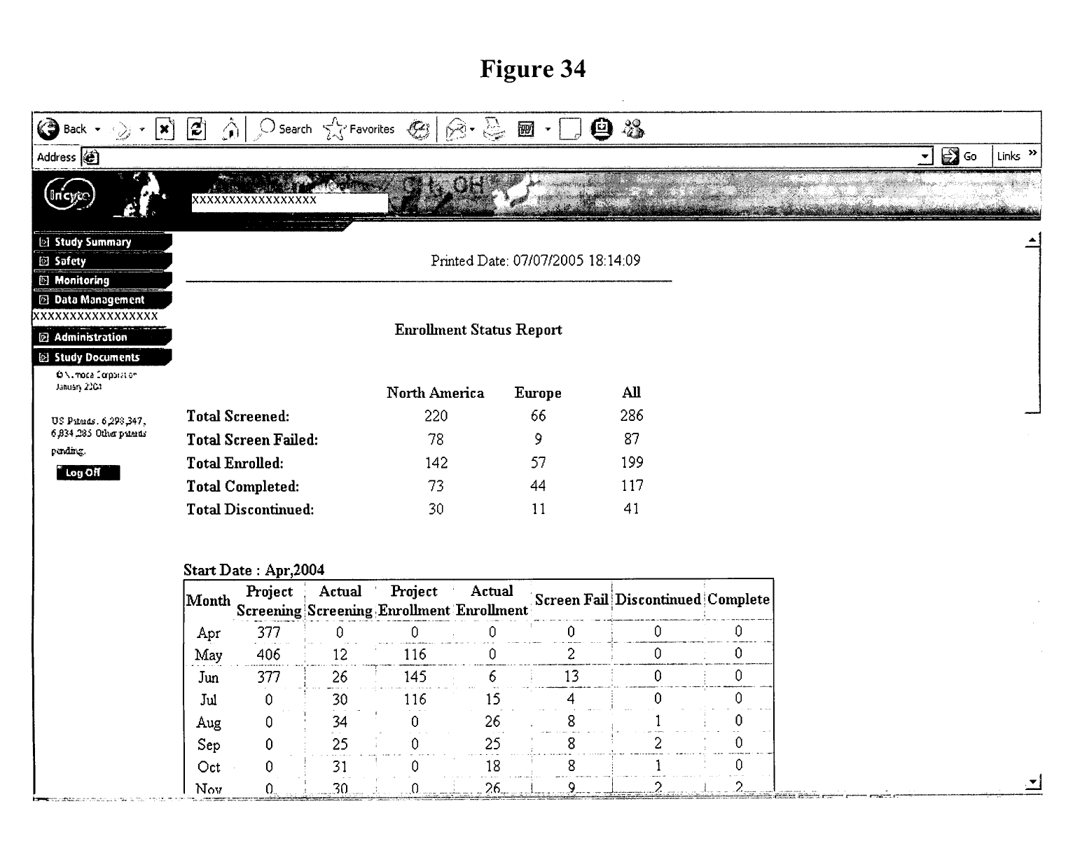

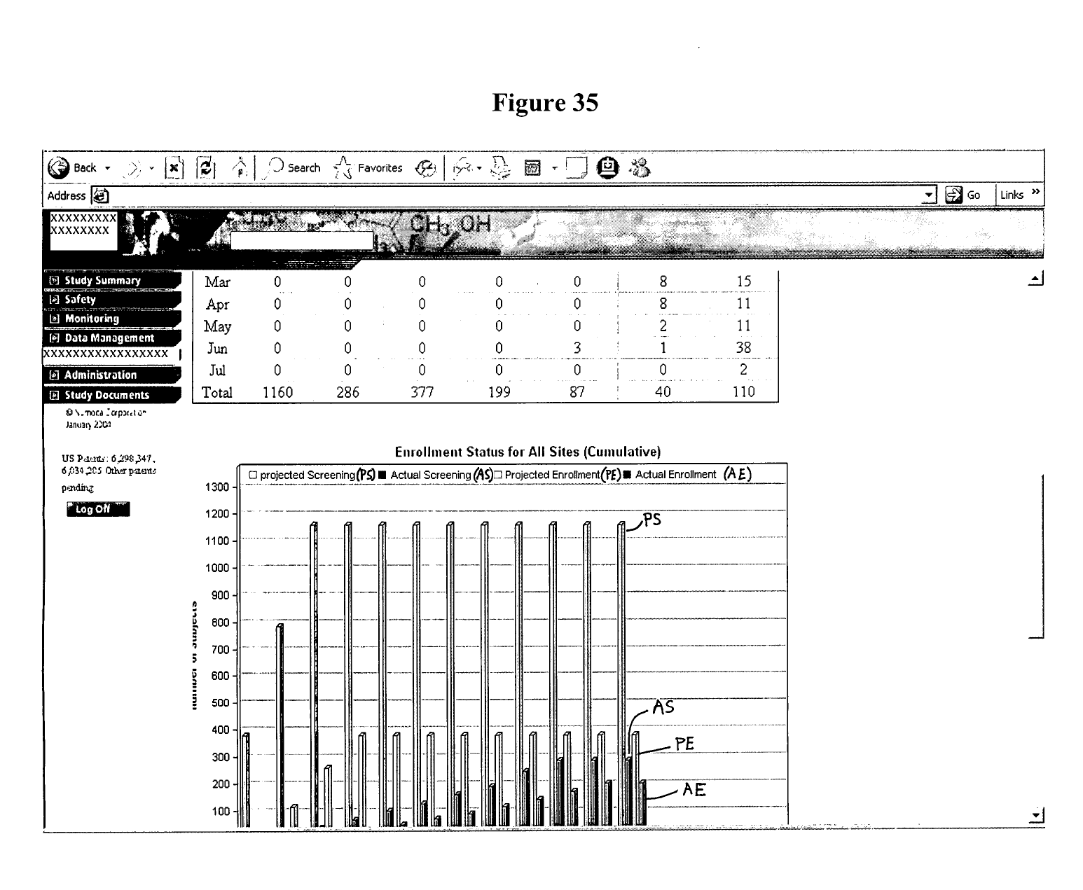

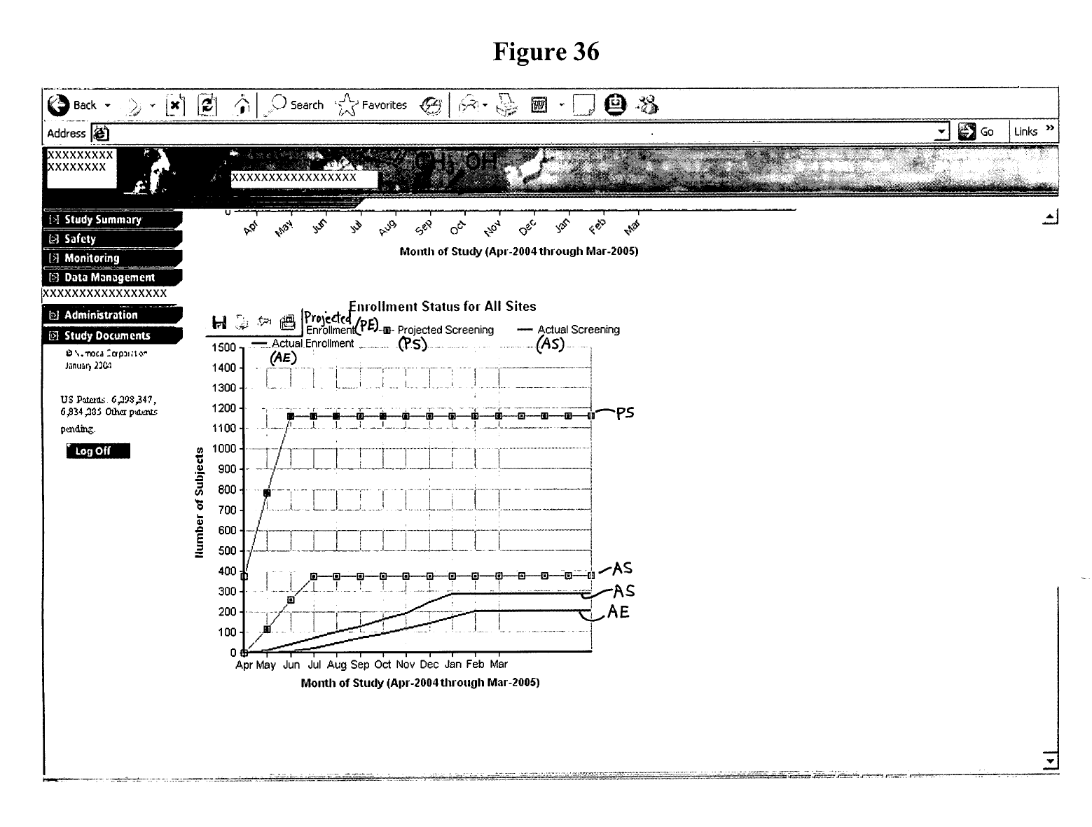

FIG. 34 is the details provided by this report that compares the projected screening against the actual screening. It also compares the projected enrollment with the actual enrollment. The present invention has used the parameters that were set for the site as shown in the present invention's set up interface in FIGS. 14 and 15. FIG. 34 shows how the present invention can then summarize that information and present it in graphic format as seen in FIG. 35. In addition, the present invention can proactively project whether the number of sites, and the speed at which they are enrolling patients, will be sufficient to completely enroll the sufficient number of patients in the time frame allotted, as seen in FIG. 36. This information lets the study team know that they need to add more sites, in order to need time deadlines. Without the present invention, the study team would need to consolidate all the information that is reported by each site and each monitor separately. The team would also need to gather information form the labs vendor and the interactive voice response vendor. They would then need to collate the information and build formulas to calculate the trends and would then need to enter this data into a program that runs these calculations and then report these numbers to the group. The present invention does all that, and does it immediately, without human intervention. The time savings and the availability of this information more quickly mean that the trial sponsor will meet the milestones for the trial. This has important financial and commercial benefits to the sponsor of the trial.

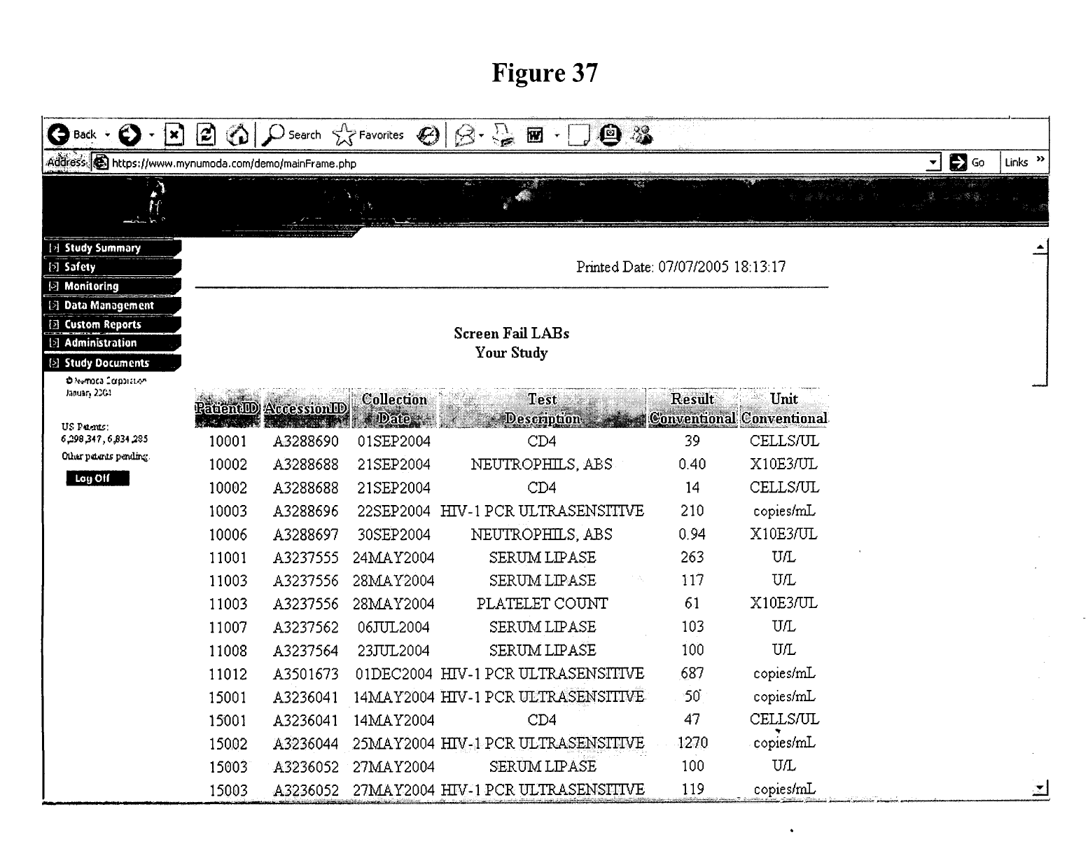

FIG. 37 shows how the present invention will report the details on the screen fail labs. This information is made available because the present invention has the parameters set for a site and versions of the protocol and all associated parameters as seen in FIGS. 6 and 7. The present invention then compares the lab results against the correct version of the protocol for each patient and provides this reports that lists the patients who have failed, along with the result. With this information, a study team can identify that the incorrect patients are being screened in the study. The team can also see if there are patients that can be eligible for a waiver. The team will also see the trend in results that signify the need for a new version of the protocol.

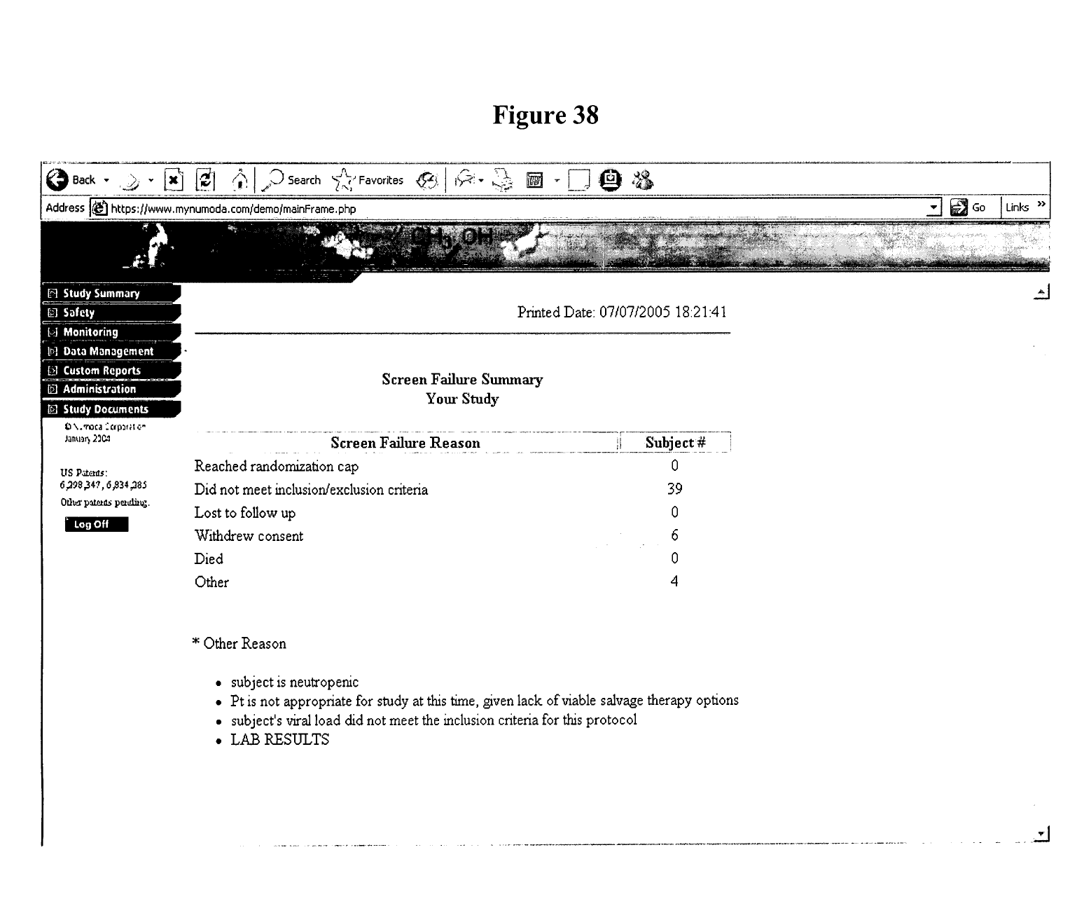

FIG. 38 shows how the present invention displays a trending report of screen failure reasons.

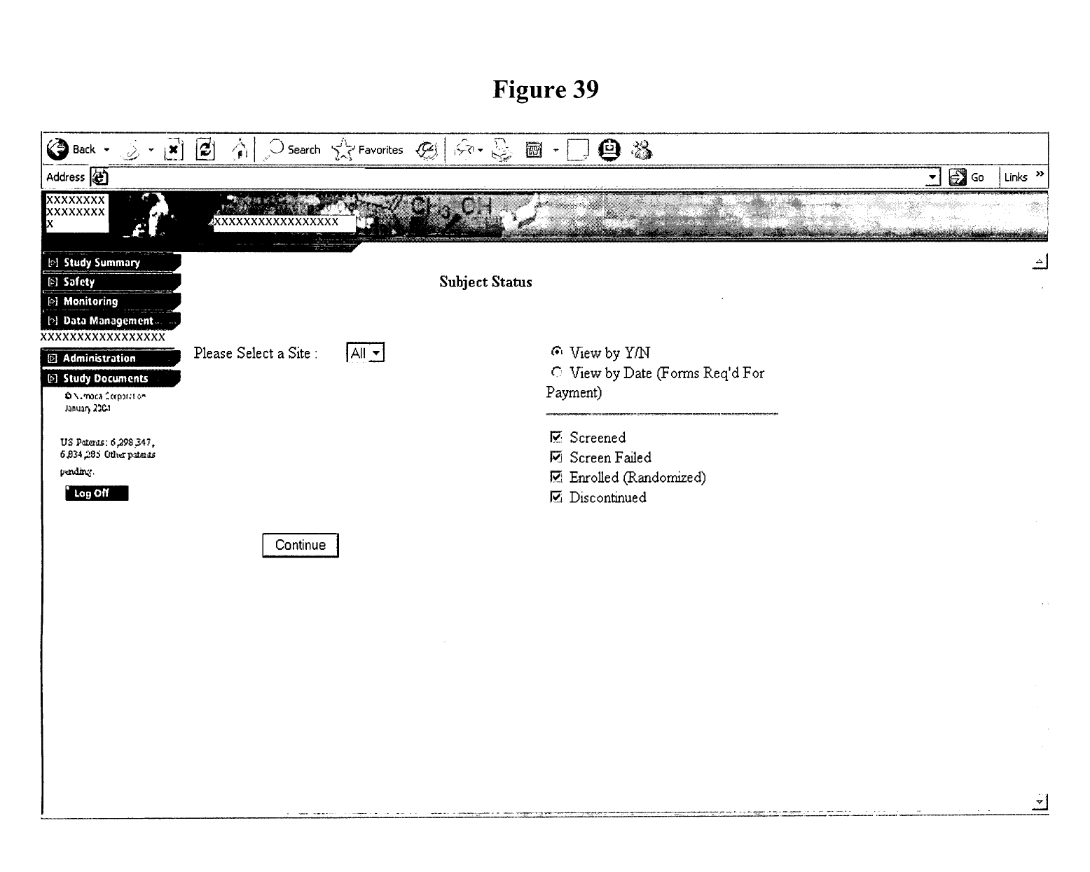

FIG. 39 shows how the present invention provides many options for the type of calculations that are done to generate a report.

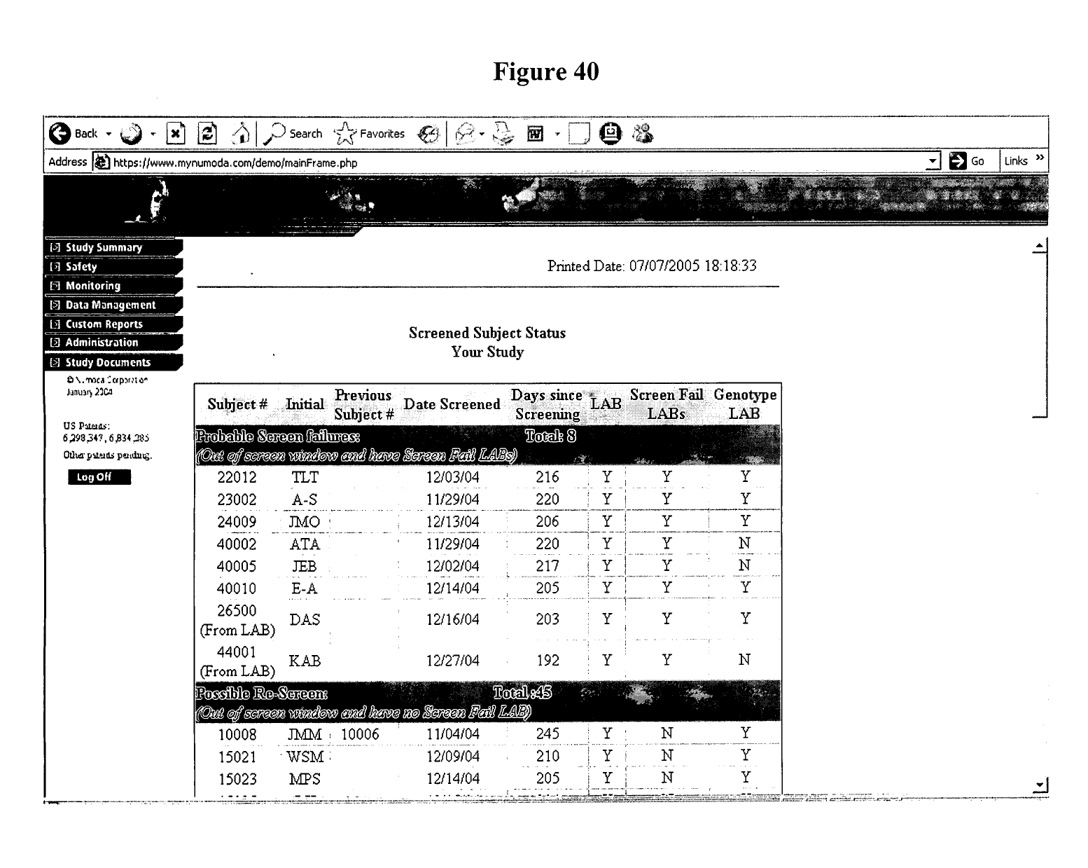

FIG. 40 is an example of how the present invention will provide suggestions for how to handle patients that are screened. This report suggests probable screen failures and gives the information why. This report also suggests possible re-screen. The present invention will process the information from a site, regarding a patient and will report if the patient is an actual screen failure, or a possible candidate for re-screening. The days since screening is displayed as well since this particular protocol requires that a patient be screened within a certain window of days. All this information assists the study team in managing and tracking the availability of patients to be enrolled for the trial. Because this information is now accessible, the present invention can calculate the actual trending of enrollment several months in advance. This advance information enables the study team to know whether a sufficient number of patients are being screened, or enrolled. If these reports prove that either screening or enrollment needs to be increased, the study team can identify the need for additional sites to be added to the study, and in what timeframe they should be added.

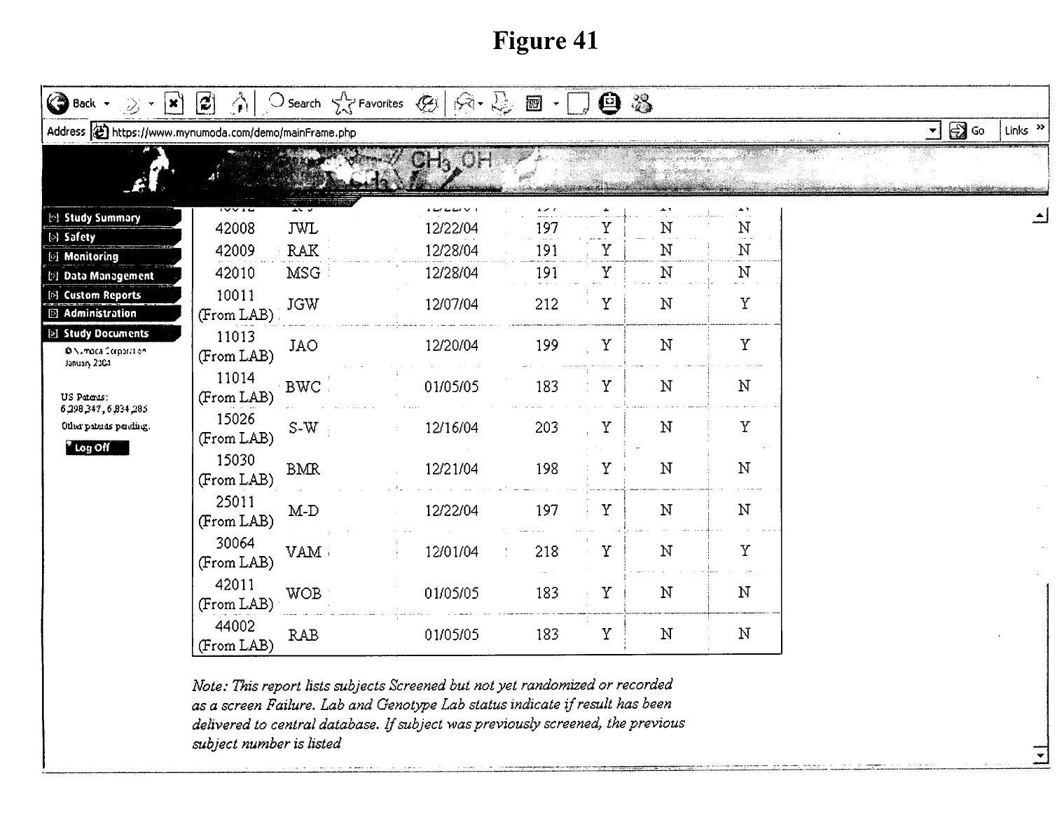

FIG. 41 is a continuation of the report that shows how the present invention pulls this information into the gateway (From LAB). There are no limitations for where the present invention can receive information to consolidate, reconcile or calculate and compare, and display.

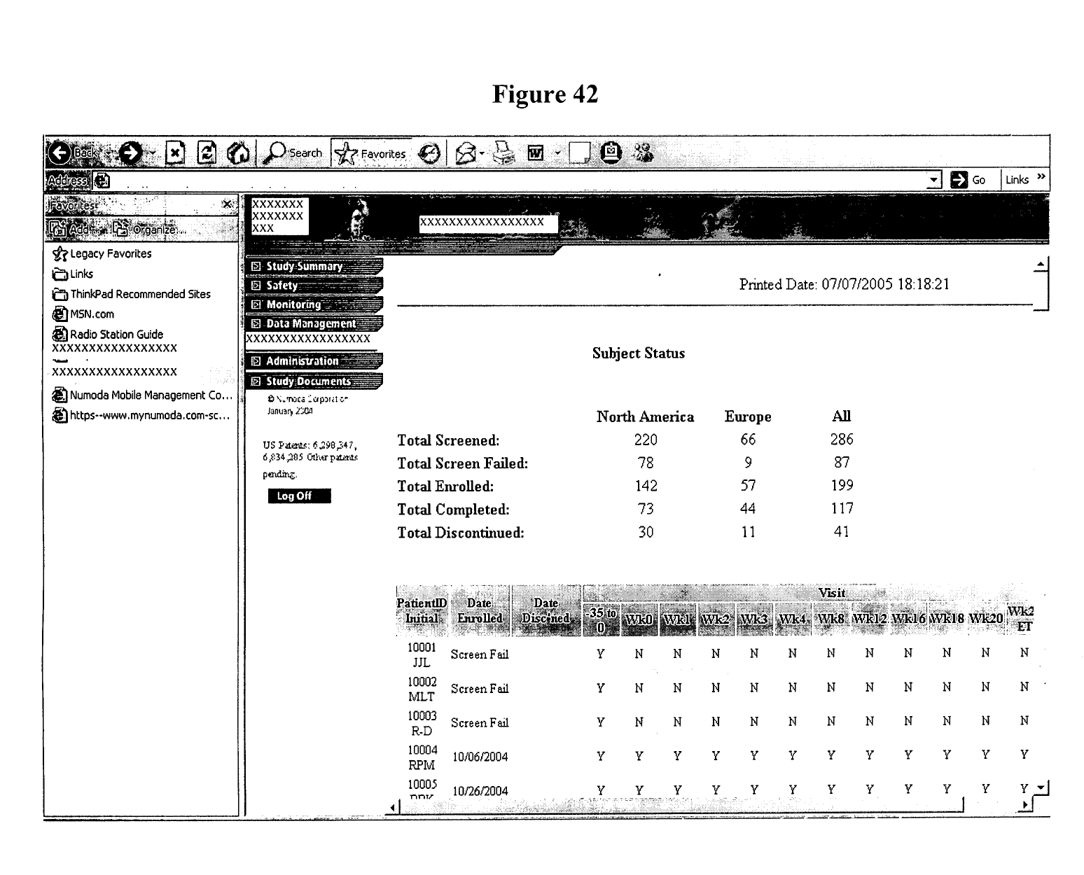

FIG. 42 is a summary report that the present invention uses and provides to show information that is used to make payments to sites for patients that are enrolled in a study. “Y” means that a payment should be made. “N” means that a payment should not be made.

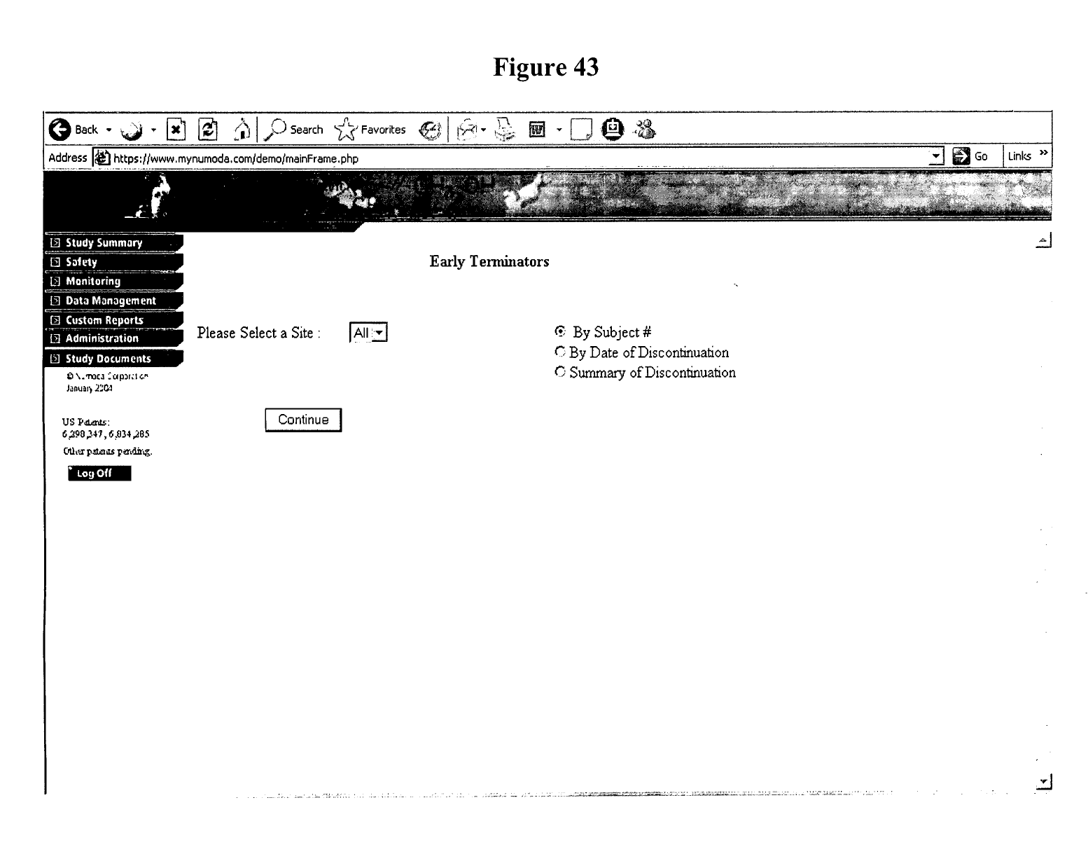

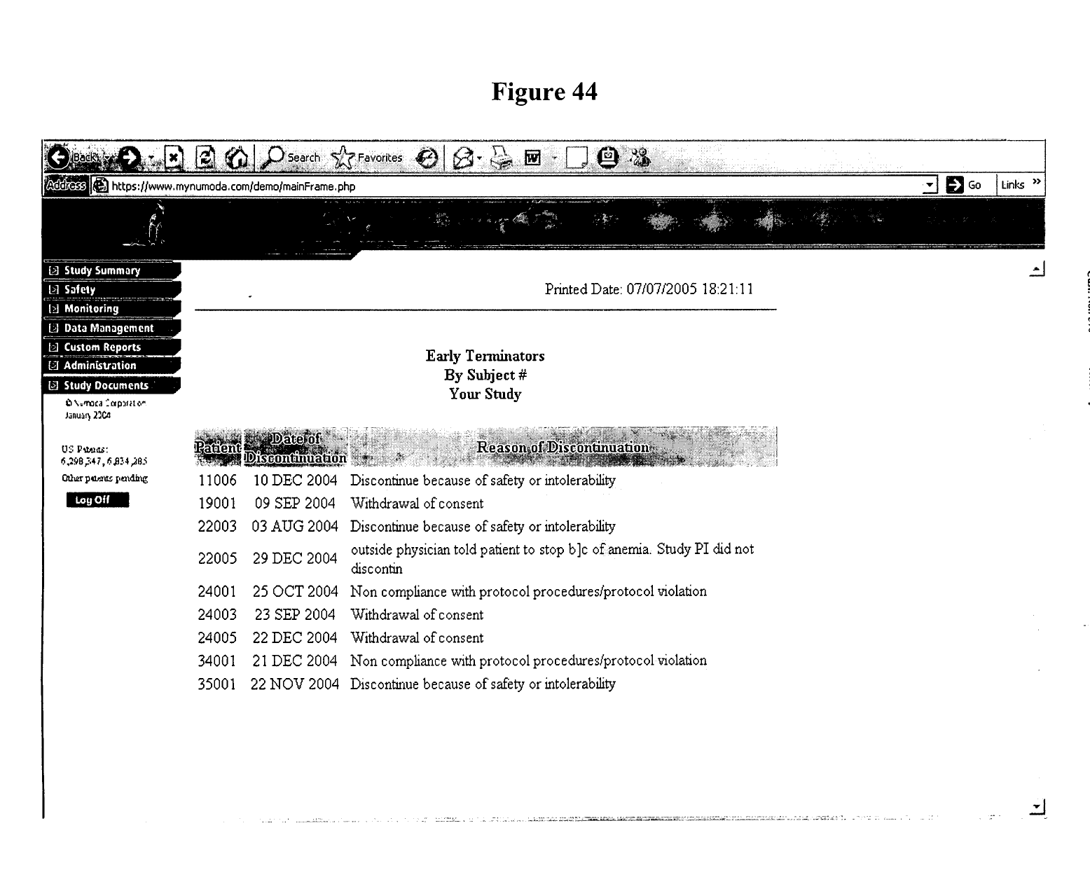

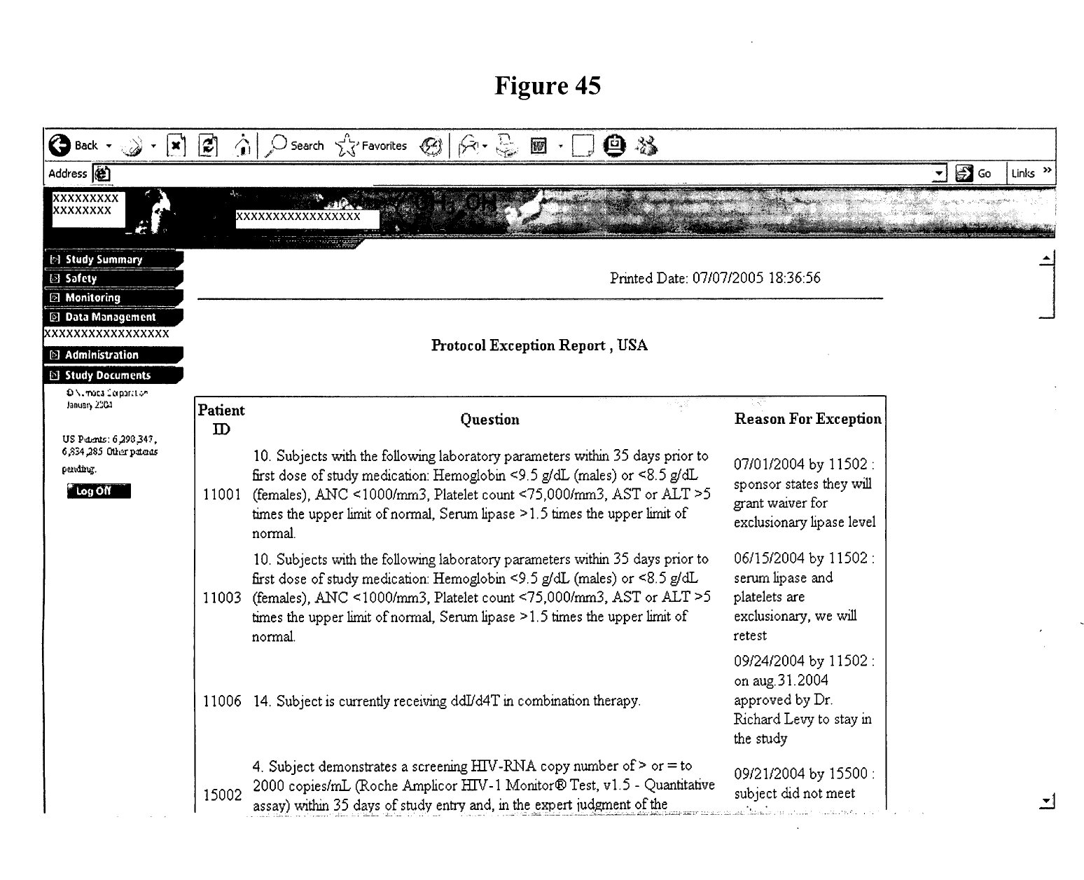

FIGS. 43 and 44 show how the present invention will provide information on Early Terminators. These are patients who quit the study or are dropped out of the study. Often these patients must be replaced and it is important to know about this and to know the details of the reason, as shown in FIG. 45.

FIG. 45 shows how the present invention provides information on protocol exceptions (waivers). Several of these waivers are given to the site since the site will be approved for a new version of the protocol that will allow these patients to be included in the study.

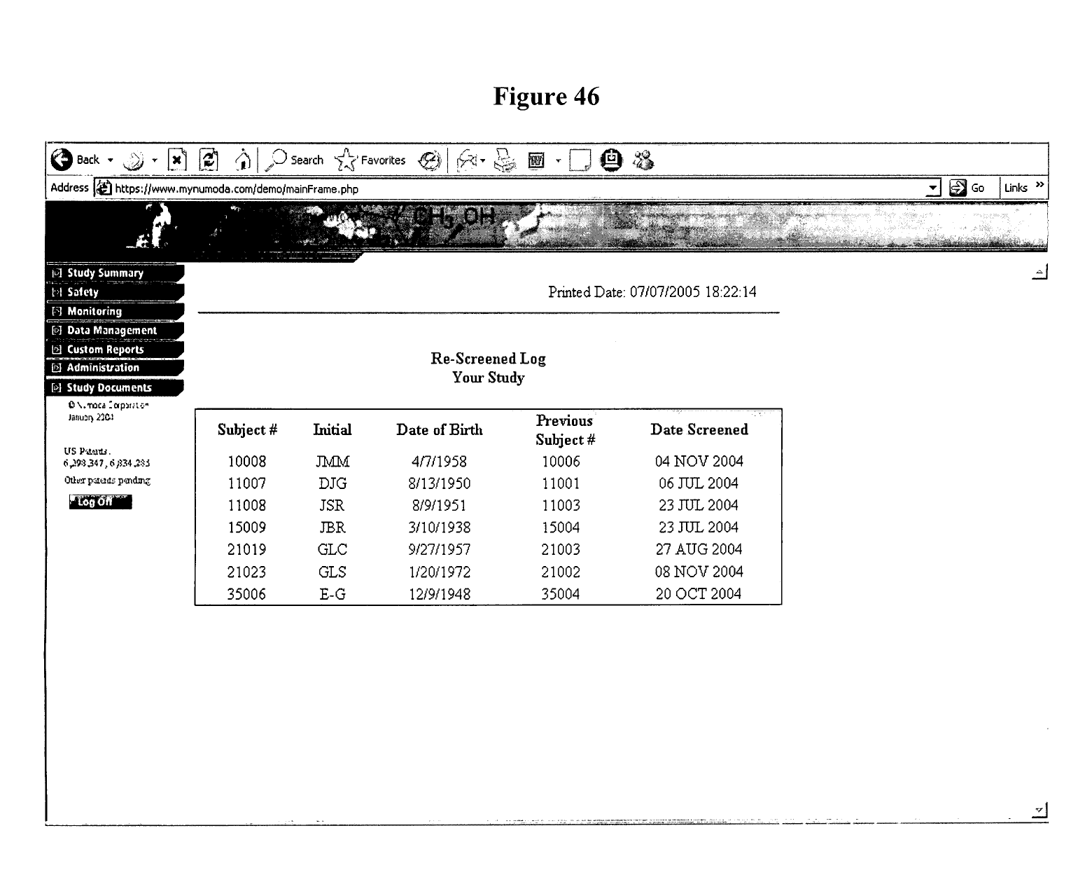

FIG. 46 shows how the present invention will provide a re-screened log. The present invention has processed all the data from the various vendors and the sites and has discovered several patients that have been brought in twice for the trial. This report is used for fraud alerts for the study team.

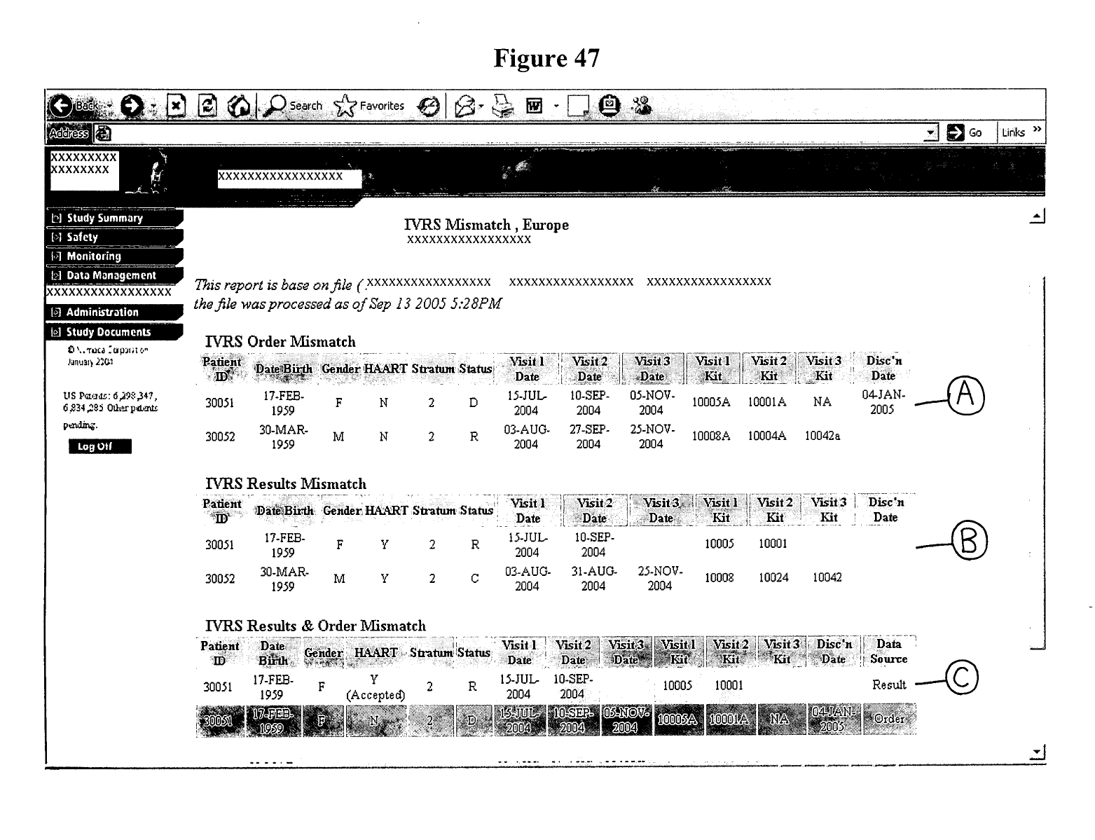

FIG. 47 shows how the present invention will process mismatches (or alerts for potentially incorrect information). In FIG. 47, the data area labeled “A” shows the IVRS orders that were brought in by the present invention gateway from the IVRS vendor. The data area labeled “B” shows the information that was brought in by the present invention gateway, from the tools at the site. See FIG. 5 for both the IVRS in FIG. 5, step C and the Pentab at the site in FIG. 5, step B. The present invention will consolidate and reconcile the data from both sources and will provide an IVRS Results and Order Mismatch report as seen in the data area labeled “C” in FIG. 47. This report instantly highlights the mismatch in data for the enrolled patient. With this information immediately available to the study team, they can focus their efforts on the source of the problem and identify patients that would otherwise not be enrolled into the trial.

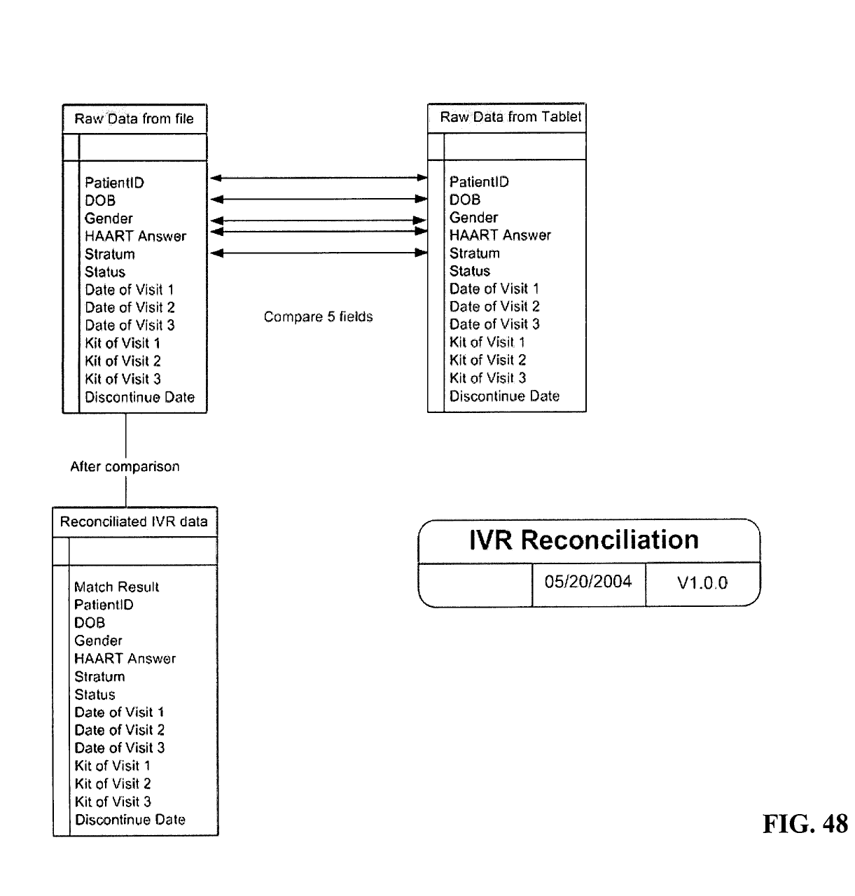

FIG. 48 is a data table structure diagram showing how the present invention compares the data brought in by two different systems and then reconciles and reports on the mismatches between the two.

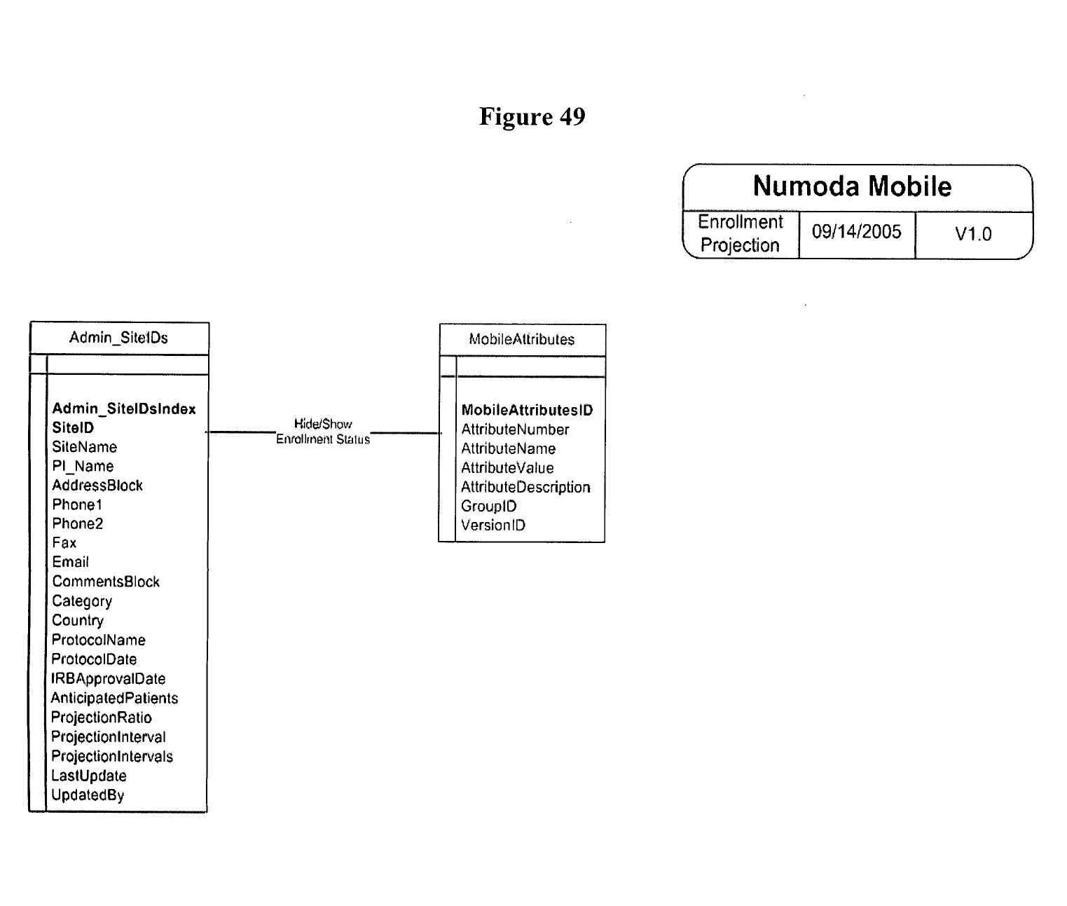

FIG. 49 is a data table structure diagram showing how the present invention handles the calculation and display of projected enrollment vs. actual enrollment for clinical investigator sites that are part of a clinical trial.

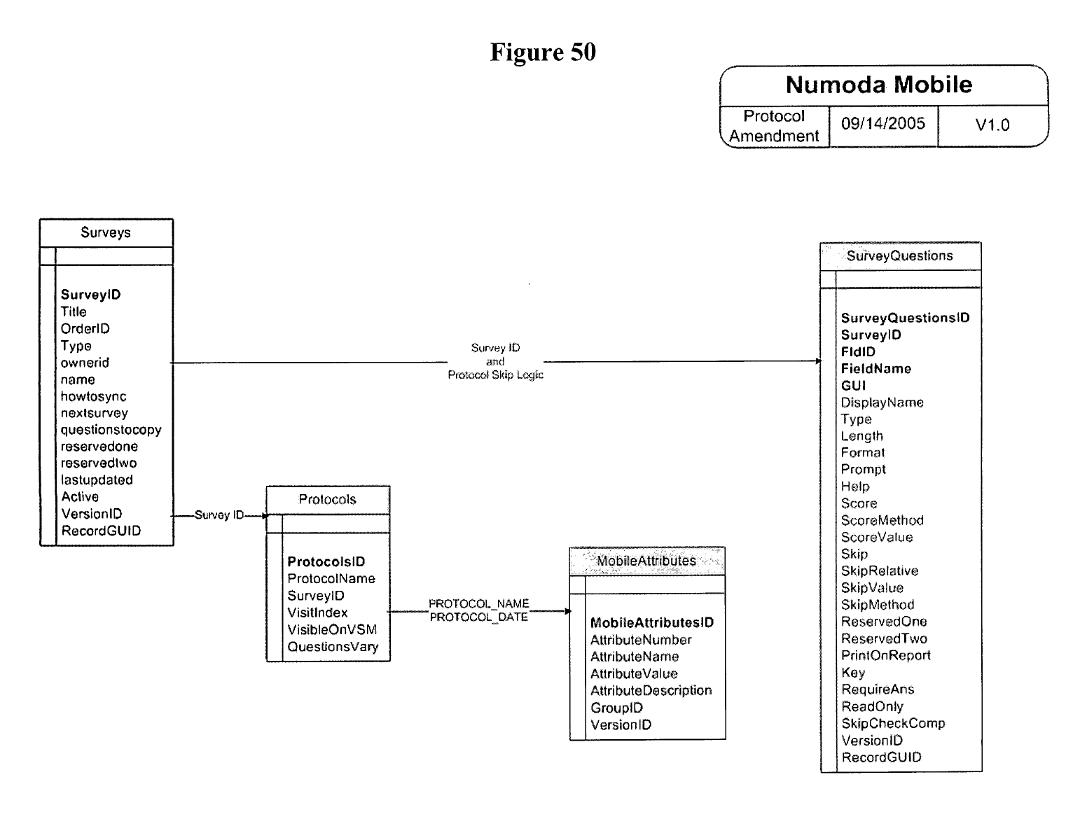

FIG. 50 is a data table structure diagram showing how the present invention handles delivering the correct version of a protocol and all accompanying screening and enrollment procedures to each site in accordance with their approval for the new version. The attached Appendix A contains the functional specifications to build the present invention and all of its components as shown on FIG. 5. These specifications are specific to a particular clinical trial protocol. However, any clinical trial protocol can be handled by the present invention.

One preferred embodiment of the present invention is described in the documentation provided in the accompanying Appendices. The Appendices include the following documents:

Appendix A: Functional Requirements Specification with Appendices I-XVIII (Site Tools)—136 pages

Appendix B: Database Specifications (Lab Gateway Specification)—9 pages

Appendix C: TAMS definition—1 page

Appendix D: Sample Results Specification (Specialty Results Specification)—1 page

Appendix E: HAART Regimen's Scope (algorithm)—7 pages

Appendix F: Central Processor Messaging Algorithms—35 pages

Appendix G: Interim Analysis Status—1 page

Appendix H: Data Transfer Guidelines Virology Database (Specialty Labs Gateway Specification)—9 pages

Appendix I: Data Transfer Guidelines (ECG Gateway Specification)—4 pages

Appendix J: HAART Drug Encoder Functional Specification—10 pages

Appendix K: System Requirements and Functional Design Specification (IVRS Gateway Specification)—4 pages

Appendix L: Medication Screening and Enrollment Tool (ARV/HAART Regimen Guidelines)—14 pages

Appendix M: RePortal Technical Specification—9 pages

The present invention may be implemented with any combination of hardware and software. If implemented as a computer-implemented apparatus, the present invention is implemented using means for performing all of the steps and functions described above.

The present invention can be included in an article of manufacture (e.g., one or more computer program products) having, for instance, computer useable media. The media has embodied therein, for instance, computer readable program code means for providing and facilitating the mechanisms of the present invention. The article of manufacture can be included as part of a computer system or sold separately.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention.

What is claimed is:

1. A computer-implemented method of tracking patient enrollment status in a clinical trial, the clinical trial having a plurality of investigative sites which perform patient screening and enrollment for the clinical trial, the method comprising:
(a) providing at each site, a user interface display screen for allowing a user at the site to enter data regarding patients who have been screened for the clinical trial and patients who have been enrolled in the clinical trial;
(b) electronically communicating the data from each of the sites to a centralized data center; and
(c) the centralized data center consolidating the data from each of the sites and providing an enrollment status report showing the total number of patients that have been screened and enrolled in the clinical trial for one or more selected time periods.

2. The method of claim 1 wherein the clinical trial further has a plurality of diagnostic sites, each diagnostic site being in electronic communication with the centralized data center, the method further comprising:
(d) electronically communicating patient data from the plurality of diagnostic sites, wherein the centralized data center further consolidates the patient data from the plurality of diagnostic sites in determining the total number of patients that have been screened and enrolled in the clinical trial.

3. The method of claim 2 wherein step (c) further comprises the central computer providing an enrollment status report showing the number of patients for a specific site that have been screened and enrolled in the clinical trial for one or more selected time periods.

4. The method of claim 1 wherein clinical trial includes a set of rules that define a properly screened patient, the method further comprising:
(d) the centralized data center using the set of rules to determine the total number of patients that have been properly screened.

5. The method of claim 1 wherein the centralized data center includes projected patient enrollment data for one or more selected time periods, and the enrollment status report further includes the projected patient enrollment data for the one or more selected time periods.

6. The method of claim 1 wherein the centralized data center includes (i) projected patient enrollment data for one or more selected time periods, (ii) data for each site regarding the projected ratio of screened to enrolled patients, and (iii) trend analysis software, the method further comprising:
(d) the centralized data center using the screening and enrollment data, the projected enrollment data, the projected ratio, and the trend analysis software to determine whether the projected enrollment for a selected time period will be met; and
(e) outputting the determination for review and potential adjustment of the enrollment and screening process.

7. A computer-implemented method of tracking patient data in a clinical trial, the clinical trial having (i) one or more investigative sites which perform patient screening and enrollment for the clinical trial, (ii) one or more diagnostic sites which perform analysis on one or more patient diagnostic tests ordered by an investigative site and generate analysis results, and (iii) a centralized data center in electronic communication with the one or more investigative sites and the one or more diagnostic sites, the method comprising:
(a) providing at each investigative site, a user interface display screen for allowing a user at the investigative site to enter data regarding patients who have been screened for the clinical trial and patients who have been enrolled in the clinical trial;
(b) electronically communicating the data from each of the investigative sites to the centralized data center;
(c) electronically communicating the analysis results from each of the diagnostic sites to the centralized data center; and
(d) the centralized data center consolidating the data and analysis results from each of the sites and providing one or more status reports regarding the patients for whom data and analysis results were received from the one or more investigative sites and the one or more diagnostic sites, wherein analysis results received by the centralized data center from a diagnostic site for specific patients are consolidated and used in the status reports regardless of whether an investigative site has communicated any data for the specific patients to the centralized data center.

8. The method of claim 7 wherein in step (d), data received by the centralized data center from the one or more investigative sites for specific patients are consolidated and used in the status reports regardless of whether a diagnostic site has communicated any data for the specific patients to the centralized data center.

9. The method of claim 7 wherein there are a plurality of investigative sites and diagnostic sites.

10. The method of claim 7 wherein one of the status reports is a mismatch report that identifies any analysis results that do not match up with any patient whose data has been entered by an investigative site.

11. The method of claim 7 wherein one of the status reports is a mismatch report that includes:
(i) any identified diagnostic tests should have been ordered by the investigative sites but which were not ordered by the investigative sites, and
(ii) any identified diagnostic tests that were ordered but which did not receive back a report of analysis results from a diagnostic site.

12. The method of claim 7 wherein the one or more status reports are based upon the results of cross-referencing the data from each of the investigative sites and the analysis results from the diagnostic sites based on a plurality of rules provided by a rules database.

13. A computer-implemented method of tracking patient screening failures in a clinical trial, the clinical trial having a plurality of investigative sites which perform patient screening and enrollment for the clinical trial, the method comprising:
(a) providing at each site, a user interface display screen for allowing a user at the site to enter data regarding patients who have been screened for the clinical trial but who were not enrolled in the clinical trial, the data including, for at least some of the patients, the reasons why the patient was not enrolled;
(b) electronically communicating the data from each of the sites to a centralized data center; and
(c) the centralized data center consolidating the data from each of the sites and providing a patient screening failure summary report showing the reasons why patients were not enrolled.

14. The method of claim 13 wherein the patient screening failure summary report further shows the total number of patients for each reason.

15. The method of claim 13 wherein the clinical trial has a plurality of inclusion/exclusion criteria, method further comprising:
(d) modifying the inclusion/exclusion criteria based on the results of the patient screening failure summary report.

16. The method of claim 13 wherein the clinical trial further has a plurality of diagnostic sites, each diagnostic site being in electronic communication with the centralized data center, the method further comprising:
(d) electronically communicating patient data from the plurality of diagnostic sites, wherein the centralized data center further consolidates the patient data from the plurality of diagnostic sites in providing the patient screening failure summary report showing the reasons why patients were not enrolled.

17. A computer-implemented method of screening and enrolling patients in a clinical trial, the clinical trial having a plurality of successively developed different protocols for defining eligibility to enroll in the clinical trial, each protocol including a set of protocol procedures, the method comprising:
(a) electronically storing each of the different protocols;
(b) entering into a computer data regarding patients who are being screened for the clinical trial, the data including patient responses to the set of protocol procedures for each of the patients; and
(c) electronically comparing in the computer the data for each patient with more than one protocol to determine whether each patient is eligible for enrollment under at least one of the developed protocols.

18. The method of claim 17 wherein each patient is screened and enrolled at a specific site, the plurality of successively developed different protocols including a current protocol that has been formally approved for a specific site, and a protocol that has not yet been formally approved for a specific site, wherein step (c) further comprises:
(i) comparing the data for each patient with the current protocol that has been formally approved for the specific site to determine whether each patient is eligible for enrollment under the formally approved current protocol for the patient's respective site, and
(ii) if the patient is not eligible for enrollment under the formally approved current protocol for the patient's respective site, comparing the data for each patient with the protocol that has not been formally approved for the specific site.

19. The method of claim 18 further comprising:
(d) if the patient is determined to be eligible for enrollment under a protocol that has not been formally approved for the specific site, automatically prompting a user via a user interface display screen to apply for a waiver to be screened and enrolled under the protocol that has not yet been formally approved for the specific site.

20. The method of claim 17 wherein the protocol procedures include inclusion/exclusion criteria.

21. The method of claim 17 wherein the protocol procedures include diagnostic tests, wherein the results of the tests must meet predefined criteria.

22. The method of claim 17 wherein at least some of the data is entered via a user interface display screen.

23. A computer-implemented method of screening and enrolling patients in a clinical trial, the clinical trial having a plurality of successively developed different protocols for defining eligibility to enroll in the clinical trial, each protocol including a set of protocol procedures, the plurality of successively developed different protocols including a current protocol that has been formally approved for a specific site, and a protocol that has not yet been formally approved for a specific site, the method comprising:
(a) electronically storing each of the protocols;
(b) entering into a computer data regarding patients who are being screened for the clinical trial, the data including patient responses to the set of protocol procedures for each of the patients;
(c) electronically comparing in the computer the data for each patient with the one or more protocols to determine whether each patient is eligible for enrollment under one of the protocols; and
(d) if the patient is not eligible for enrollment under the current protocol that has been formally approved for the patient's site, but is eligible under the protocol that has not yet been formally approved for the patient's site, identifying the patient as potentially eligible for enrollment under the not yet formally approved protocol.

24. The method of claim 23 further comprising:
(e) automatically prompting a user via a user interface display screen to apply for a waiver to be screened and enrolled under the protocol that has not yet been formally approved for the specific site.

25. The method of claim 23 wherein the protocol procedures include inclusion/exclusion criteria.

26. The method of claim 23 wherein the protocol procedures include diagnostic tests, wherein the results of the tests must meet predefined criteria.

27. A computer-implemented method of tracking diagnostic tests conducted in a screening stage of a clinical trial, wherein a protocol having inclusion/exclusion criteria is defined for eligibility to enroll in the clinical trial, the inclusion/exclusion criteria including one or more diagnostic tests that must be performed, the clinical trial having (i) one or more investigative sites which perform patient screening and enrollment for the clinical trial, including ordering of the diagnostic tests, (ii) one or more diagnostic sites which perform analysis on one or more of the ordered diagnostic tests, and (iii) a centralized data center in electronic communication with the one or more investigative sites and the diagnostic sites, the method comprising:
(a) automatically identifying one or more diagnostic tests to be performed for the patients from the inclusion/exclusion criteria associated with the respective patients;
(b) automatically tracking at the centralized data center:
(i) whether the one or more investigative sites ordered the identified diagnostic tests, and
(ii) whether the one or more diagnostic sites delivered analysis results to the centralized data center for the identified and ordered diagnostic tests; and
(c) automatically generating mismatch reports at the centralized data center that include:
(i) any identified diagnostic tests that were not ordered by the investigative sites, and
(ii) any identified diagnostic tests that were ordered but which did not receive back a report of analysis results from a diagnostic site.

28. The method of claim 27 wherein step (a) further includes automatically identifying the completion target dates of the respective one or more diagnostic tests, the mismatch reports in step (c)(i) further includes any identified diagnostic tests that were not ordered by the investigative sites by a first predefined date, and the mismatch reports in step (c)(ii) further includes any identified diagnostic tests that were ordered but which did not receive back a report of analysis results from a diagnostic site by a second predefined date, wherein the first and second predefined dates are calculated from the completion target dates.

29. The method of claim 27 further comprising:
(d) viewing the mismatch reports via a user interface display screen.

30. A computer-implemented method of tracking diagnostic tests conducted in a screening stage of a clinical trial, wherein a protocol having inclusion/exclusion criteria is defined for eligibility to enroll in the clinical trial, the inclusion/exclusion criteria including (i) one or more diagnostic tests that must be performed, the clinical trial having one or more investigative sites which perform patient screening and enrollment for the clinical trial, including ordering of the diagnostic tests, and (ii) a centralized data center in electronic communication with the one or more investigative sites, the method comprising:
(a) automatically identifying one or more diagnostic tests to be performed for the patients from the inclusion/exclusion criteria associated with the respective patients;
(b) automatically tracking at the centralized data center whether the one or more investigative sites ordered the identified diagnostic tests; and
(c) automatically generating mismatch reports at the centralized data center that include any identified diagnostic tests that were not ordered by the investigative sites.

31. A computer-implemented method of tracking diagnostic tests conducted in a screening stage of a clinical trial, wherein a protocol having inclusion/exclusion criteria is defined for eligibility to enroll in the clinical trial, the inclusion/exclusion criteria including one or more diagnostic tests that must be performed, the clinical trial having (i) one or more investigative sites which perform patient screening and enrollment for the clinical trial, including ordering of the diagnostic tests, (ii) one or more diagnostic sites which perform analysis on one or more of the ordered diagnostic tests, and (iii) a centralized data center in electronic communication with the one or more investigative sites and the diagnostic sites, the method comprising:
(a) automatically identifying one or more diagnostic tests to be performed for the patients from the inclusion/exclusion criteria associated with the respective patients;
(b) automatically tracking at the centralized data center whether the one or more diagnostic sites delivered analysis results to the centralized data center for the identified and ordered diagnostic tests; and
(c) automatically generating mismatch reports at the centralized data center that include any identified diagnostic tests that were ordered but which did not receive back a report of analysis results from a diagnostic site.
